(19) United States
(12) Patent Application Publication
(10) Pub. No.: US 2024/0182421 A1
(43) Pub. Date: Jun. 6, 2024

(54) SYNTHESIS OF ENDOCHIN-LIKE QUINOLONES

(71) Applicants: Oregon Health & Science University, Portland, OR (US); The United States Government as Represented by the Department of Veterans Affairs, Washington, DC (US)

(72) Inventors: Michael K. Riscoe, Portland, OR (US); Aaron Nilsen, Portland, OR (US); Haihong Jin, Portland, OR (US); Sovitj Pou, Portland, OR (US); Rolf W. Winter, Portland, OR (US); Rozalia Ana Dodean, Damascus, OR (US); Katherine M. Liebman, Portland, OR (US)

(73) Assignees: Oregon Health & Science University, Portland, OR (US); The United States Government as Represented by the Department of Veterans Affairs, Washington, DC (US)

(21) Appl. No.: 18/547,129

(22) PCT Filed: Mar. 17, 2022
(86) PCT No.: PCT/US2022/020830

§ 371 Date: Aug. 18, 2023

Related U.S. Application Data
(60) Provisional application No. 63/163,284, filed on Mar. 19, 2021.
(60) Provisional application No. 63/213,587, filed on Jun. 22, 2021.
(60) Provisional application No. 63/221,442, filed on Jul. 13, 2021.

(51) Int. Cl. C07D 215/233

(52) U.S. Cl. CPC C07D 215/233

(57) ABSTRACT

Described herein are new synthetic routes for production of Endochin-Like Quinolone (ELQ) compounds. Synthetic routes to 3-substituted 4(1H)-quinolones are presented that are amenable to industrial scale preparation. One herein presented approach is relatively short, does not require palladium, and involves no chromatographic separation. A second herein presented approach is similarly relatively short, does not require palladium, involves no chromatographic separation, and also avoids high vacuum distillation. Additionally, both approaches require no protecting group chemistry because the insoluble 4(1H)-quinolone is not formed until the final reaction step.

### CROSS-REFERENCE TO RELATED APPLICATIONS

This application is the 371 National Phase of International Application No. PCT/US22/20830, filed Mar. 17, 2022, which claims priority to and the benefit of the earlier filing of U.S. Provisional Application No. 63/163,284, filed on Mar. 19, 2021; U.S. Provisional Application No. 63/213,587, filed on Jun. 22, 2021; and U.S. Provisional Application No. 63/221,442, filed on Jul. 13, 2021, each of which is incorporated by reference herein in its entirety.

### STATEMENT OF GOVERNMENT SUPPORT

This invention was made with government support under R01 Al100569 and R01 Al141412 awarded by The National Institutes of Health and W81XWH-19-2-0031 awarded by the Department of Defense. The government has certain rights in the invention.

### FIELD

The present invention concerns new synthetic steps, routes, and intermediates useful in the production of endochin-like quinolone compounds that are useful in the treatment of protozoal infections, such as malaria, toxoplasmosis, babesiosis, amoebiasis, giardiasis, leishmaniasis, trypanosomiasis, coccidiosis, and schistosomiasis.

### BACKGROUND OF THE DISCLOSURE

When endochin (FIG. 1) was first synthesized in 1940, its high activity in the avian malaria model led to hope that a new and promising class of antimalarials had been discovered. Endochin and a series of derivatives were tested against malaria in humans, but the results were disappointing and the compounds were not pursued further as antimalarials (Salzer et al., Chemische Berichte 1948, 81 (1), 12-19). This compound class was reexamined in the early 2000s, when a series of new endochin derivatives were discovered that were curative of patent malaria in mice. The outstanding representatives of this new generation of Endochin-Like Quinolones (ELQs) bore a methyl group at position 2, a diphenyl ether substituent at position 3, and additional substituents in the second ring of the quinolone system (FIG. 1).

These ELQs demonstrated high antimalarial potency in vitro and in vivo, parasite selectivity, chemical and metabolic stability, desirable pharmacokinetics and low mammalian cell toxicity. In addition to their antimalarial activity, compounds in the series were later found to be highly active against other Apicomplexa, for which satisfactory treatments are urgently needed. These include various Babesia species (affecting humans, cattle, horses, and dogs; Silva et al., Parasit Vectors 2020, 13 (1), 606), Theileria equi (horses; Silva et al., Parasit Vectors 2020, 13 (1), 606), Neospora caninum (cattle, sheep, goats, deer, horses, dogs; Muller et al., Int J Parasitol 2017, 47 (12), 801-809), Besnoitia besnoiti (cattle; Eberhard et al., Front Vet Sci 2020, 7, 96), and Toxoplasma gondii (humans, cats, marine mammals; Doggett et al., Proc Natl Acad Sci U S A 2012, 109 (39), 15936-41; McConnell et al., ACS Infect Dis 2018, 4 (11), 1574-1584; Dubey & Jones, Int J Parasitol 2008, 38 (11), 1257-78; Shapiro et al., Proc Biol Sci 2019, 286(1909), 20191334). Finally, an ELQ compound has been found to have a potent, low-dose inhibitory effect on the nematode Echinococcus multilocularis, a fox-transmitted tapeworm that may be fatal to its hosts, including humans (Rufener et al., Int J Parasitol Drugs Drug Resist 2018, 8 (3), 440-450).

With its favorable properties and broad activity, the ELQ compound class may yield effective, safe treatments for a range of important human and animal afflictions. Of these, malaria is a particularly serious and prevalent human disease. In 2019 alone, it afflicted 229 million people worldwide and caused 409,000 deaths (World malaria report 2020: 20 years of global progress and challenges; World Health Organization: Geneva, 2020). ELQ-300, in the form of prodrug ELQ-331, has recently been accepted as a preclinical candidate by the Medicines for Malaria Venture for potential use in the prevention and treatment of malaria (Frueh et al., ACS Infect Dis 2017, 3 (10), 728-735). ELQ-316 and its prodrugs, on the other hand, have the greatest potency against Toxoplasma gondii and Babesia microti. Toxoplasmosis may have infected up to one third of all humankind; this infection can be serious for immunocompromised individuals, and can cause harm to the fetus when contracted during pregnancy (Alday et al., Drug Des Devel Ther 2017, 11, 273-293, Bigna et al., Sci Rep 2020, 10 (1), 12102). Babesiosis affects both humans and livestock and is, together with neosporosis and besnoitiosis, a significant problem for livestock husbandry (Silva et al., Parasit Vectors 2020, 13 (1), 606; Muller et al., Int J Parasitol 12017, 47 (12), 801-809; Eberhard et al., Front Vet Sci 2020, 7, 96). If compounds in this series are to become practical drug candidates, a synthetic route is needed that is amenable to industrial scale production.

Synthetic routes for the preparation of endochin-like quinolone compounds can be seen in U.S. Pat. Nos. 8,598,354 (Riscoe et al.), 9,206,131 (Riscoe et al.), 10,532,983 (Riscoe et al.), 10,584,098 (Riscoe et al.), and U.S. Patent Publication No. 2017/127820 (Manetsch et al.). Additional descriptions are seen in the articles Quinolone-3-diarylethers: a new class of antimalarial drug, Nilsen et al., Science Translational Medicine, 5(177): 177ra37 (2013); Nilsen et al., J Med Chem., 7(9):3818-3834 (2014); Cross et al., J Med Chem., 57(21): 8860-8879 (2014); and Monastryrskyi et al., J Organic Chem, 80(5): 2513-2520 (2015); Namelikonda et al., Euro J Organic Chem., 23: 3328-3334 (2017).

It is desirable to identify synthetic route(s) amenable to large scale production. Historically, 3-alkyl substituted 4(1H)-quinolones have been synthesized by reaction of a substituted β-keto ester with an aniline via the Conrad-Limpach reaction (Scheme 1) (Salzer et al., Chemische Berichte 1948, 81 (1), 12-19; Winter et al., Exp Parasitol 2008, 118 (4), 487-97; Winter et al., Exp Parasitol 2011, 127 (2), 545-51). Endochin itself is easily synthesized by the Conrad-Limpach reaction, a sequence of two reactions. In the first stage, 2-n-heptylacetoacetic ester is condensed with meta-anisidine to form a β-anilinocrotonate; in the second stage, thermal cyclisation produces endochin, usually in DOWTHERM™ A heat transfer fluid boiling at 250° C. Of the two isomers formed in the second step, endochin, the 7-methoxy isomer, crystallizes out of the reaction mixture upon cooling, while the 5-methoxy isomer remains dissolved. The yield of endochin is around 40-50% with this procedure (Salzer et al., Chemische Berichte 1948, 81 (1), 12-19; Winter et al., Exp Parasitol 2011, 127 (2), 545-51; Winter et al., Exp Parasitol 2008, 118 (4), 487-97).

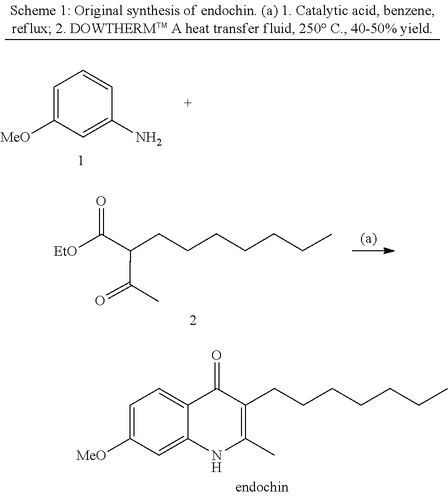 [SMILES: COc1cccc(N)c1 ; CCCCCCCC(C(C)=O)C(=O)OCC ; CCCCCCCc1c(C)[nH]c2cc(OC)ccc2c1=O]

Alternative cyclization methods for 4(1H)-quinolones have also been explored (Torii et al., Tetrahedron Letters 1990, 31 (49), 7175-7178; Pidathala et al., J Med Chem 2012, 55 (5), 1831-43; Ho et al., ACS Catalysis 2019, 9 (1), 504-510). 3-Aryl substituted 4(1H)-quinolones, such as ELQ-300, have been synthesized by reaction of a 4(1H)-quinolone with a reactive aryl moiety (Ward et al., Tetrahedron Lett 2009, 50 (47), 6494-9497; Cross et al., J Org Chem 2010, 75(24), 8654-7; Ravi et al., J Org Chem 2015, 80 (10), 5369-76), primarily via a Suzuki-Miyaura reaction (Ward et al., Tetrahedron Lett 2009, 50 (47), 6494-9497; Cross & Manetsch, J Org Chem 2010, 75(24), 8654-7; Ravi et al., J Org Chem 2015, 80 (10), 5369-76). However, 4(1H)-quinolones are known to be sparingly soluble in organic solvents and water, and are difficult to isolate using chromatography. As a result, reactions that produce a mixture of 3-aryl-4(1H)-quinolones have proven difficult to separate and generally give poor yields of pure 4(1H)-quinolone products.

3-Diaryl ether 4(1H)-quinolones, such as 6-chloro-7-methoxy-2-methyl-3-(4-(4-(trifluoromethoxy)phenoxy)phenyl)quinolin-4(1H)- one (ELQ-300), have been synthesized by reaction of a ‘protected’ 3-halo-4(1H)-quinolone with a diaryl ether boronic ester via the Suzuki-Miyaura reaction (Doggett et al., Proc Natl Acad Sci U S A 2012, 109 (39), 15936-41; Nilsen et al., Sci Transl Med 2013, 5 (177), 177ra37; Nilsen et al., J Med Chem 2014, 57 (9), 3818-34; Cross et al., J Med Chem 2014, 57 (21), 8860-79). Conversion of 3-halo-4(1H)-quinolones to their corresponding 4-ethoxy-3-halo quinolines provided a ‘protected’ 4(1H)-quinolone intermediate that: 1) performed well in Suzuki-Miyaura reactions, 2) was sufficiently soluble in the reaction solvent, dimethyl formamide, 3) could be isolated via chromatography, and 4) was readily converted back to a 4(1H)-quinolone.

The original route to ELQ-300, involving in parallel the formation of the 4(1H)-quinolone 7 via a Conrad-Limpach reaction (Scheme 2, reaction c) and the formation of the diaryl ether side chain 4 via an Ullmann reaction (reaction a), was designed to allow for late-stage structural variation at the 3-position using a Suzuki-Miyaura reaction (reaction f). The synthesis presented in Scheme 2 is a version of the originally published synthesis that was optimized for a larger scale. We discovered that conversion of 3-halo-4(1H)-quinolone 8 to the corresponding 4-ethoxy-3-halo quinoline 9 provided a protected 4(1H)-quinolone intermediate that performed well in a Suzuki-Miyaura reaction and provided a product 10 that could be isolated by chromatography and then readily converted back to the desired 4(1H)-quinolone, such as ELQ-300 (Doggett et al., Proc Natl Acad Sci U S A 2012, 109 (39), 15936-41; Nilsen, et al., Sci Transl Med 2013, 5 (177), 177ra37; Nilsen et al., J Med Chem 2014, 57 (9), 3818-34; Cross et al., J Med Chem 2014, 57 (21), 8860-79). However, though this route has been used to prepare hundreds of grams of various ELQ compounds, it is not ideal for industrial scale production because it is relatively long (seven reaction steps), requires the use of a somewhat expensive palladium catalyst, and involves a high vacuum distillation step (compound 12), at least two chromatographic separations (compounds 5 and 10), and multiple recrystallizations.

Additional complications arose when performing large scale versions of the reactions shown in Scheme 2. The original iodination reaction (iodine, potassium iodide and n-butylamine in DMF) did not scale well, so it was replaced with reaction (d) that required at least 72 hours and did not progress much beyond 96% completion. During the formation of the 4-O-ethyl ether (reaction e), N-ethylation occurred, resulting in a small amount of an N-ethylated side product that was difficult to separate. Unwanted reduction of the 3-iodo group during the Suzuki reaction (f) resulted in a small amount of reduced side product that was also difficult to separate using chromatography and was inseparable after the quinolone was re-formed in reaction (g). Finally, removal of the 4-O-ethyl ether protecting group (reaction g) requires relatively harsh reaction conditions (HBr in acetic acid (AcOH) at 90° C.) and long reaction times (>48 hours), which can result in the demethylation of the 7-OMe ether.

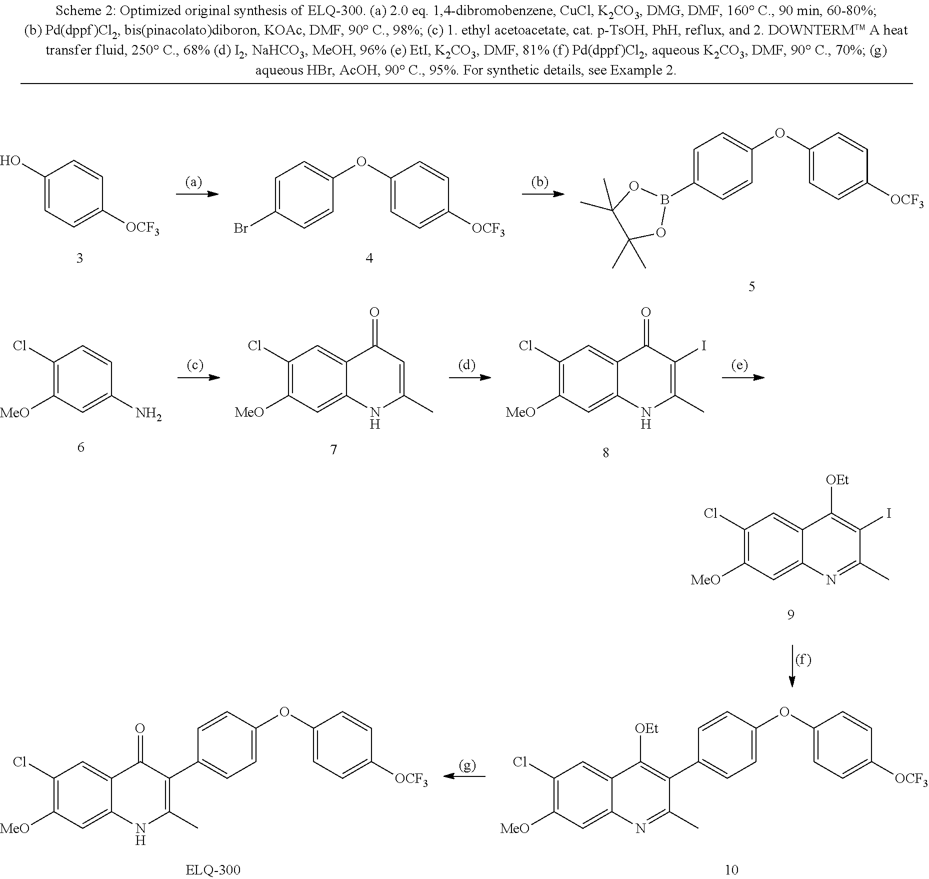

Recently, the synthesis of 3-diaryl ether 4(1H)-quinolones via substituted β-keto esters has been revisited (Monastyrskyi et al., J Org Chem 2015, 80 (5), 2513-2520; Namelikonda et al., Euro J Org Chem. 2017, 2017 (23), 3328-3334). However, these routes were not shown to be amenable to industrial scale production.

There remains a need for synthetic schemes for the preparation of endochin-like quinolone compounds, particularly those that may be practicable for industrial-scale production.

### BRIEF DESCRIPTION OF THE DRAWINGS

FIG. 1 provides structures of endochin, ELQ-300, ELQ-316 and ELQ-331.

FIG. 2 illustrates synthesis of ethyl 2-(4-(4-(trifluoromethoxy)phenoxy)phenyl)acetate 12 using the herein-described method that does not require high vacuum distillation. Reaction (a): CuCl, K₂CO₃, DMG DMF, 160° C.; 2-APDTC; Reaction (b): EtOH/H⁺, reflux, 2-3 h.

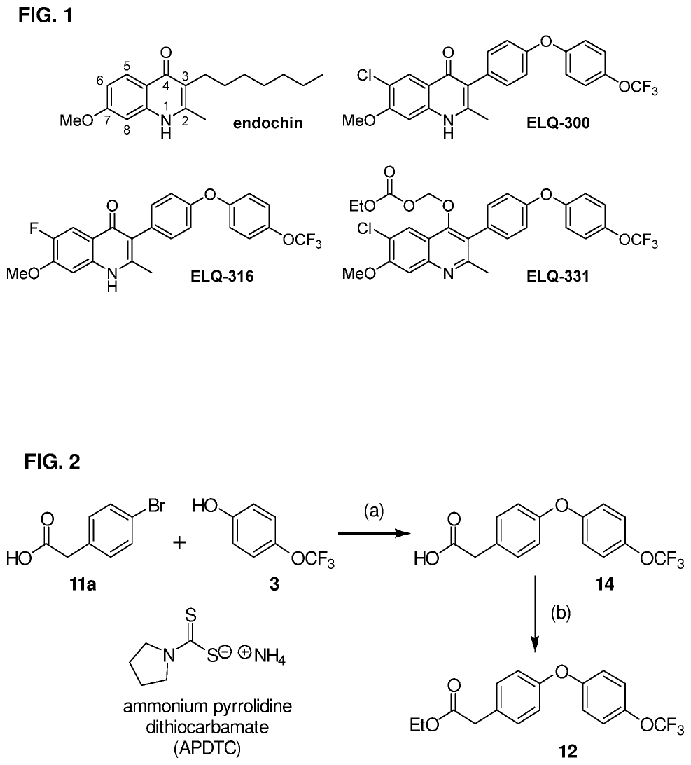

### SUMMARY OF THE DISCLOSURE

Herein, a synthetic route to 3-substituted 4(1H)-quinolones is presented that is amenable to industrial scale preparation. A first herein presented optimized approach is relatively short (4 reaction steps), does not require palladium, and involves no chromatographic separation. A second herein presented optimized approach is similarly relatively short (5 reaction steps), does not require palladium, involves no chromatographic separation, and also avoids high vacuum distillation. Additionally, both approaches require no protecting group chemistry because the insoluble 4(1H)-quinolone is not formed until the final reaction step.

An embodiment provides a method for the preparation of a compound of Formula (I):

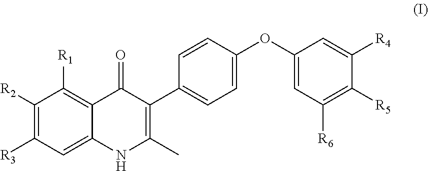

wherein:
R₁ is selected from the group of H, F, Cl, Br, CN, —CH₂F, —CHF₂, —CF₃, —OCH₃, —OCH₂F, —OCHF₂, —OCF₃, —SF₅, and —CH₃;
R₂ is selected from the group of H, F, Cl, Br, CN, —CH₂F, —CHF₂, —CF₃, —OCH₃, —OCH₂F, —OCHF₂, —OCF₃, —SF₅, and —CH₃;
R₃ is selected from the group of H, F, Cl, Br, CN, —CH₂F, —CHF₂, —CF₃, —OCH₃, —OCH₂F, —OCHF₂, —OCF₃, —SF₅, and —CH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃;
   the method involving the steps of:
a) reacting an optionally substituted phenol compound of Formula (A)

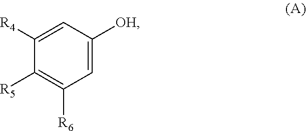

wherein R₄, R₅, and R₆ are as defined above, with an alkyl 2-(4-bromophenyl) acetate compound of the Formula (B)

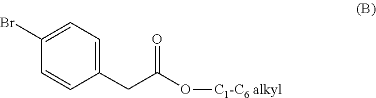

to produce an optionally substituted alkyl 2-(4-phenoxyphenyl)acetate compound of Formula (C)

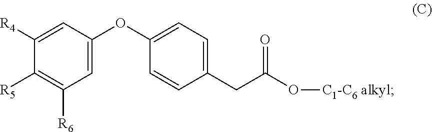

b) treating the compound of Formula (C) with a non-nucleophilic base and acetic anhydride to prepare a compound of Formula (D)

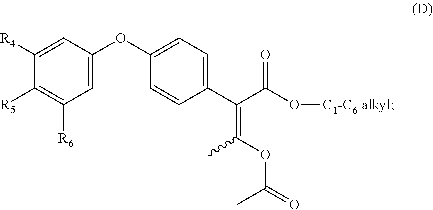

c) treating the compound of Formula (D) with an acid to prepare a compound of Formula (E)

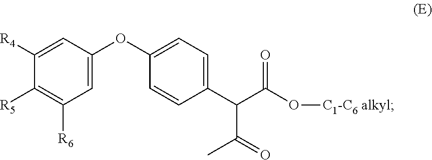

d) reacting the compound of Formula (E) with an optionally substituted aniline compound of Formula (F)

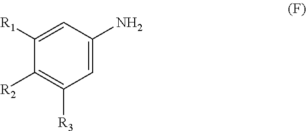

to prepare a compound of Formula (G)

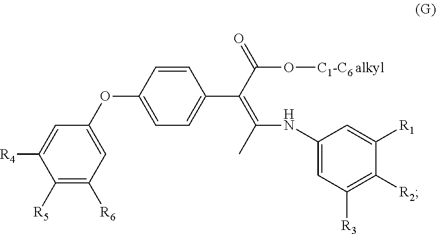

and
e) heating the compound of Formula (G) at a temperature of from about 150° C. to about 300° C. to prepare the compound of Formula (I).

Additional embodiments involve, respectively, the individual reactions of steps a), b), c), d) and e) described above.

Yet another embodiment is a method for the preparation of a compound of Formula (I):

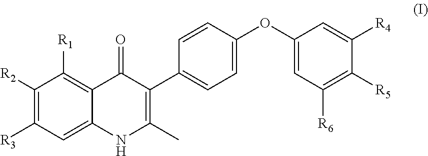

wherein:
R₁ is selected from the group of H, F, Cl, Br, CN, —CH₂F, —CHF₂, —CF₃, —OCH₃, —OCH₂F, —OCHF₂, —OCF₃, —SF₅, and —CH₃;
R₂ is selected from the group of H, F, Cl, Br, CN, —CH₂F, —CHF₂, —CF₃, —OCH₃, —OCH₂F, —OCHF₂, —OCF₃, —SF₅, and —CH₃;
R₃ is selected from the group of H, F, Cl, Br, CN, —CH₂F, —CHF₂, —CF₃, —OCH₃, —OCH₂F, —OCHF₂, —OCF₃, —SF₅, and —CH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃;
   the method involving the steps of:
a) reacting an optionally substituted phenol compound of Formula (A)

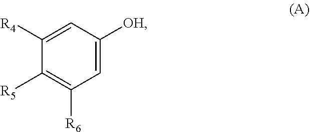

wherein R₄, R₅, and R₆ are as defined above, with 4-bromophenylacetic acid, in the presence of a copper catalyst, to form intermediate diaryl ether carboxylic acid (H)

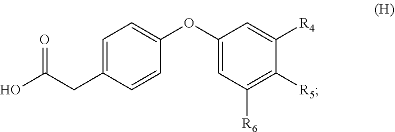

b) removing the copper catalyst, for instance using ammonium pyrrolidine dithiocarbamate (APDTC);
c) esterifying the diaryl ether carboxylic acid (H) to form an optionally substituted alkyl 2-(4-phenoxyphenyl)acetate compound of Formula (C)

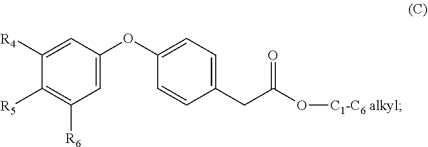

d) treating the compound of Formula (C) with a non-nucleophilic base and acetic anhydride to prepare a compound of Formula (D)

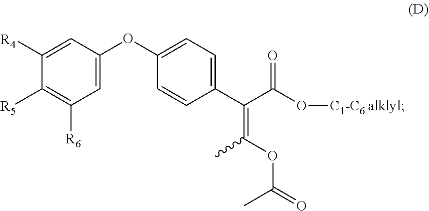

e) treating the compound of Formula (D) with an acid to prepare a compound of Formula (E)

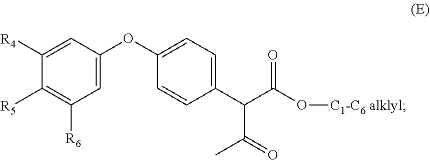

f) reacting the compound of Formula (E) with an optionally substituted aniline compound of Formula (F)

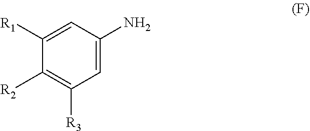

to prepare a compound of Formula (G)

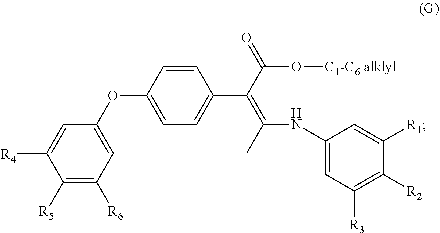

and
g) heating the compound of Formula (G) at a temperature of from about 150° C. to about 300° C. to prepare the compound of Formula (I).

Also provided is an embodiment that is a method of preparing a compound of Formula (C):

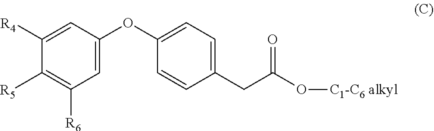

the method involving:
reacting, in the presence of a copper catalyst, 4-bromophenylacetic acid (11a) with a phenol moiety substituted by R₄, R₅, and R₆, as defined herein, to form the intermediate diaryl ether carboxylic acid (H);
removing the copper catalyst; and
esterifying the carboxylic acid (H) to Compound (C).

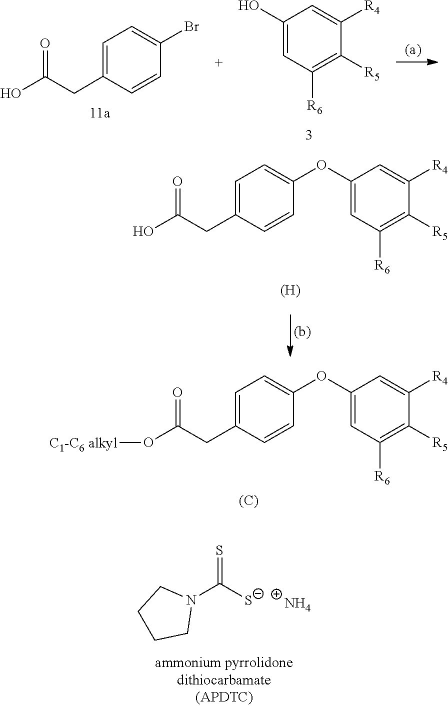

Yet another method embodiment is a method of preparing Compound (C), involving: reacting 4-bromophenylacetic acid (11a) with a phenol moiety substituted by R₄, R₅, and R₆ (3), as defined herein, in a first medium involving a copper catalyst to form a second medium involving the intermediate diaryl ether carboxylic acid (H) and the copper catalyst;
a) removing the copper catalyst from the second medium with a copper chelating agent to create a third medium involving the intermediate diaryl ether carboxylic acid (H); and
b) reacting the intermediate diaryl ether carboxylic acid (H) in the third medium with a C₁-C₆ alkanol (for instance, ethanol) to form esterified Compound (C).

Also provided are methods of synthesis of ethyl 2-(4-(4-(trifluoromethoxy)phenoxy)phenyl)acetate 12, which methods do not involve high vacuum distillation.

### DETAILED DESCRIPTION OF THE DISCLOSURE

In some embodiments, the compound of Formula (I) prepared by the steps provided in the Summary of the Disclosure is one wherein:
R₁ is selected from the group of H and F;
R₂ is selected from the group of H, F, and Cl;
R₃ is selected from the group of H, F, and —OCH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃.

Provided herein is a method of preparing a compound of Formula (C):

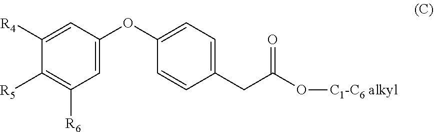

the method involving reacting an optionally substituted phenol compound of Formula (A)

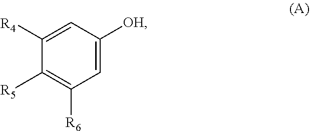

wherein R₄, R₅, and R₆ are as defined above, with an alkyl 2-(4-bromophenyl) acetate compound of the Formula (B)

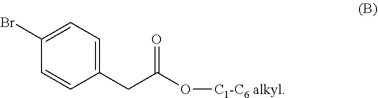

In some embodiments, the coupling reaction of the compounds of Formula (A) and Formula (B) to prepare a compound of Formula (C), seen in step a), above, may be accomplished in an organic solvent in the presence of a) a base; b) a catalyst involving copper, a copper salt, or a hydrate thereof; and c) a ligand selected from the group of an amino acid or a salt thereof, an acetic acid derivative, a phosphinite, a phosphonate, an imine, a diamine, an oxime, an oxime ether, and a diketone.

In some embodiments, the C₁-C₆ alkyl group in the compound of Formula (B) is selected from the group of ethyl, isopropyl, and t-butyl.

Non-limiting examples of copper agents, in addition to elemental copper, that may be used include copper iodide (Cul), copper bromide (CuBr), copper chloride (CuCl), CuCl₂·H₂O, copper powder, copper trifluomethanesulfonate (Cu(OTf)₂), copper II acetate (Cu(OAc)₂), and copper II sulfate (Cu(SO₄)).

Non-limiting organic solvents useful in step a) include N,N-dimethylformamide (DMF), tetrahydrofuran (THF), dimethyl sulfoxide (DMSO), acetonitrile, toluene, and 1,4-dioxane.

Non-limiting examples of bases that may be used include N,N-diisopropylethylamine (DIPEA), triethyl amine (Et₃N), potassium carbonate (K₂CO₃), sodium carbonate (Na₂CO₃), rubidium carbonate (Rb₂CO₃), cesium carbonate (Cs₂CO₃), and potassium phosphate (K₃PO₄)

Non-limiting ligands useful in step a) include N,N-dimethylglycine, 3-(dimethylamino)propanoic acid, 2-(pyridin-2-yl)acetic acid, 8-((di(furan-2-yl)phosphaneyl)oxy)quinoline, 8-((dicyclohexylphosphaneyl)oxy) quinoline, and (1E,1′E)-N,N′-(ethane-1,2-diyl)bis(1-(thiophen-2-yl)methanimine). Additional ligands for use in the methods herein may be seen in the article Screening of ligands for the Ullmann synthesis of electron-rich diaryl ethers: Beilstein J. Org. Chem., Otto et al., 8, 1105-1111, 2012 (doi:10.3762/bjoc.8.122).

The reaction of step a) may be conducted at an effective temperature of from about 100° C. to about 220° C. In some embodiments the reaction is conducted at a temperature of from about 120° C. to about 200° C. In other embodiments the reaction is conducted at a temperature of from about 140° C. to about 180° C. In still other embodiments the reaction is conducted at a temperature of from about 150° C. to about 170° C.

Another embodiment provides a method for the preparation of compound of Formula (D)

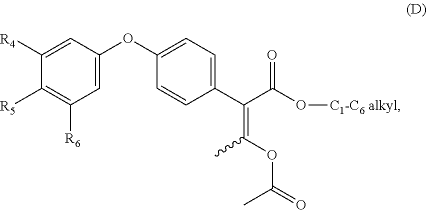

wherein R₄, R₅, and R₆ are as defined above, the method involving treating the compound of Formula (C), above, with a non-nucleophilic base and acetic anhydride.

Non-limiting examples of non-nucleophilic bases that may be used in the reaction immediately above and in step b) of the process in the Summary of the Invention include lithium bis(trimethylsilyl)amide (LiHMDS), lithium diisopropylamide (LDA), lithium tert-butoxide, sodium bis(trimethylsilyl)amide (NaHMDS), lithium diisopropylamide (LDA), and potassium bis(trimethylsilyl)amide (KHMDS).

Non-limiting organic solvents useful in step b) include N,N-dimethylformamide (DMF), tetrahydrofuran (THF), dimethyl sulfoxide (DMSO), acetonitrile, toluene, and 1,4-dioxane.

The reaction of the compound of Formula (C) to prepare the compound of Formula (D) may be accomplished in some embodiments at a temperature of from about −40° C. to about 0° C. In other embodiments, the reaction may be accomplished at a temperature of from about −30° C. to about −10° C. In additional embodiments, the reaction may be accomplished at a temperature of from about −25° C. to about −15° C.

A further embodiment provides a method of preparing a compound of Formula (E)

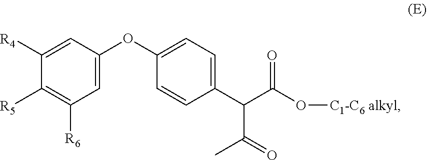

the method involving reacting a compound of Formula (D), above, with an acid.

Non-limiting examples of acids that may be used in the preparation of the compound of Formula (E) include p-toluenesulfonic acid, methanesulfonic acid, dry hydrobromic acid, dry hydrochloric acid, trifluoromethanesulfonic acid, trifluoroacetic acid, sulfuric acid, benzenesulfonic acid, and ethanesulfonic acid, optionally dissolved in acetic acid or another suitable solvent.

In one embodiment, the acid used in the preparation of the compound of Formula (E) is about 5% to about 25% p-toluenesulfonic acid, or the monohydrate thereof, in acetic acid.

Non-limiting examples of solvents that may be used in the preparation of the compound of Formula (E) include acetic acid, dichloromethane, chloroform, carbon tetrachloride, tertiary butyl methyl ether, diisopropyl ether, tetrahydrofuran, cyclohexane, diethyl ether, benzene, toluene, xylene, and acetic anhydride.

Another embodiment provides a method to prepare a compound of Formula (G)

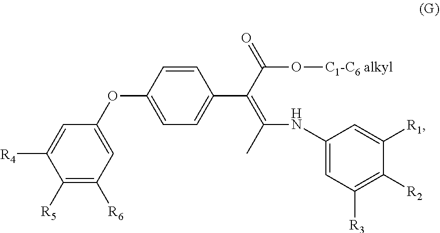

wherein R₁, R₂, R₃, R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; the method involving reacting the compound of Formula (E), as seen above, with an optionally substituted aniline compound of Formula (F)

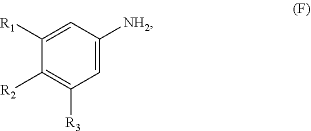

wherein R₁, R₂, and R₃ are as defined above.

A further embodiment provides the method of preparing a compound of Formula (G), as described above, wherein R₁ is selected from the group of H and F; R₂ is selected from the group of H, F, and Cl; R₃ is selected from the group of H, F, and —OCH₃; and each of R₄, R₅, and R₆ is independently selected from the group of H, F, Cl, Br, CN, —CH₂F, —CHF₂, —CF₃, —OCH₃, —OCH₂F, —OCHF₂, —OCF₃, —SF₅, and —CH₃.

A further embodiment provides a method of preparing a compound of Formula (I)

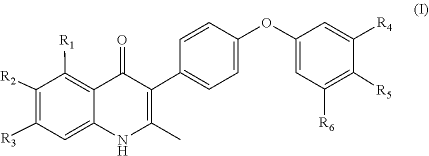

wherein: each of R₁, R₂, R₃, R₄, R₅, and R₆ is selected from the group of H, F, Cl, Br, CN, —CH₂F, —CHF₂, —CF₃, —OCH₃, —OCH₂F, —OCHF₂, —OCF₃, —SF₅, and —CH₃; the method involving treating a compound of Formula (G), as defined above, to a temperature of from about 180° C. to about 270° C. for a period of time effective in converting the compound of Formula (G) to the compound of Formula (I).

In some embodiments the compound of Formula (I) is prepared by treating the compound of Formula (G) to a temperature of from about 150° C. to about 300° C., or from about 200° C. to about 300° C., for an effective period of time. In other embodiments the compound of Formula (I) is prepared by treating the compound of Formula (G) to a temperature of from about 200° C. to about 270° C., or from about 210° C. to about 260° C., for an effective period of time. In further embodiments the compound of Formula (I) is prepared by treating the compound of Formula (G) to a temperature of from about 210° C. to about 260° C. for an effective period of time. In additional embodiments the compound of Formula (I) is prepared by treating the compound of Formula (G) to a temperature of from about 240° C. to about 260° C. for an effective period of time. In still other embodiments the compound of Formula (I) is prepared by treating the compound of Formula (G) to a temperature of from about 245° C. to about 255° C. for an effective period of time.

In some embodiments, the preparation of the compound of Formula (I) from the compound of Formula (G) is accomplished in the presence of a heat transfer fluid.

The effective period of time for this reaction step is understood to be conversion of an acceptable amount of the treated compound of Formula (G) to the compound of Formula (I) for the applicable scientific, industrial, financial, or other consideration at hand. In some embodiments, the effective time is the amount of time required to obtain a desired percentage yield of the compound of Formula (I). The effective period of time for this step will depend upon the reaction volume, as well as the temperature and/or the heat transfer fluid utilized. In some embodiments the effective time will be 5 hours or less. In some embodiments the effective time will be one hour or less. In other embodiments, the effective time will be from about 15 minutes to about one hour. In still other embodiments, the effective time will be from about 20 minutes to about 40 minutes.

Within each of the individual embodiments listed above for the method of preparing the compound of Formula (I), there is a further embodiment involving the steps, conditions, and materials of the earlier embodiment, wherein R₁ is selected from the group of H and F; R₂ is selected from the group of H, F, and Cl; R₃ is selected from the group of H, F, and —OCH₃; and each of R₄, R₅, R₆ is independently selected from the group of H, F, Cl, Br, CN, —CH₂F, —CHF₂, —CF₃, —OCH₃, —OCH₂F, —OCHF₂, —OCF₃, —SF₅, and —CH₃.

### Novel Intermediates

An embodiment provides a compound of Formula (C-1):

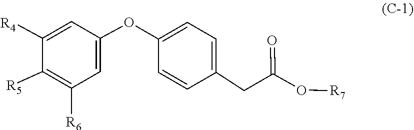

wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl; with the proviso that, when R₇ is methyl, at least one of R₄, R₅, and R₆ is not H.

An additional embodiment provides a compound of Formula (C-1) wherein R₄, R₅, and R₆ are each independently selected from the group of H, F, Cl, Br, CN, —CH₂F, —CHF₂, —CF₃, —OCH₂F, —OCH₃, —OCHF₂, —OCF₃, —SF₅, and —CH₃; and R₇ is C₁-C₄ alkyl; with the proviso that, when R₇ is methyl, at least one of R₄, R₅, and R₆ is not H.

Another embodiment provides a compound of Formula (C-1) wherein R₄, R₅, and R₆ are each independently selected from the group of H, F, Cl, Br, CN, —CH₂F, —CHF₂, CF₃, —OCH₃, —OCH₂F, —OCHF₂, —OCF₃, —SF₅, and —CH₃; and R₇ is C₁-C₃ alkyl; with the proviso that, when R₇ is methyl, at least one of R₄, R₅, and R₆ is not H.

A further embodiment provides a compound of Formula (C-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

A further embodiment provides a compound of Formula (C-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₅ alkyl.

A further embodiment provides a compound of Formula (C-1) wherein R₅ is —OCF₃; R₄, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

A still further embodiment provides a compound of Formula (C-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

A still further embodiment provides a compound of Formula (C-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

Another embodiment provides a compound of Formula (C-1) wherein R₅ is —OCF₃; R₄ and R₆ are each H; and R₇ is C₁-C₆ alkyl.

Another embodiment provides a compound of Formula (C-1) wherein R₅ is —OCF₃; R₄ and R₆ are each H; and R₇ is C₁-C₄ alkyl.

Another embodiment provides a compound of Formula (C-1) wherein R₅ is —OCF₃; R₄ and R₆ are each H; and R₇ is C₁-C₃ alkyl.

Another embodiment provides a compound of Formula (C-1) wherein R₅ is —OCF₃; R₄ and R₆ are each H; and R₇ is C₁-C₂ alkyl.

In a further embodiment involves a compound of Formula (C-1a):

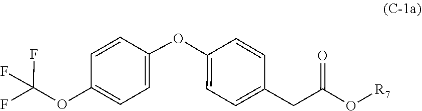

wherein R₇ is C₁-C₆ alkyl.

Another embodiment involves a compound of Formula (C-1a) wherein R₇ is C₁-C₄ alkyl. Still another embodiment involves a compound of Formula (C-1a) wherein R₇ is C₁-C₃ alkyl.

Another embodiment involves a compound of Formula (C-1a) wherein R₇ is C₁-C₄ alkyl. Still another embodiment involves a compound of Formula (C-1a) wherein R₇ is C₁-C₂ alkyl.

In one embodiment, the compound of Formula (C-1) is ethyl 2-(4-(4-(trifluoromethoxy)phenoxy)phenyl)acetate (12):

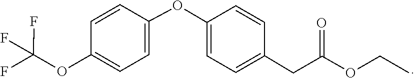 [SMILES: CCOC(=O)Cc1ccc(Oc2ccc(OC(F)(F)F)cc2)cc1]

Also provided is a compound of Formula (D-1):

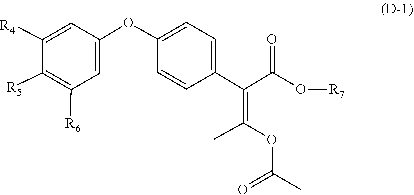

wherein: R₄, R₅, and R₆ are each independently selected from the group of H, F, Cl, Br, CN, —OCH₃, —OCH₃, —CH₂F, —CHF₂, —CF₃, —SF₅, and —CH₃; and R₇ is C₁-C₆ alkyl.

An additional embodiment provides a compound of Formula (D-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

Another embodiment provides a compound of Formula (D-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

Another embodiment provides a compound of Formula (D-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

A further embodiment provides a compound of Formula (D-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

A further embodiment provides a compound of Formula (D-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₅ alkyl.

A further embodiment provides a compound of Formula (D-1) wherein R₅ is —OCF₃; R₄, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

A still further embodiment provides a compound of Formula (D-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

A still further embodiment provides a compound of Formula (D-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

Another embodiment provides a compound of Formula (D-1) wherein R₅ is —OCF₃; R₄ and R₆ are each H; and R₇ is C₁-C₆ alkyl.

Another embodiment provides a compound of Formula (D-1) wherein R₅ is —OCF₃; R₄ and R₆ are each H; and R₇ is C₁-C₄ alkyl.

Another embodiment provides a compound of Formula (D-1) wherein R₅ is —OCF₃; R₄ and R₆ are each H; and R₇ is C₁-C₃ alkyl.

Another embodiment provides a compound of Formula (D-1) wherein R₅ is —OCF₃; R₄ and R₆ are each H; and R₇ is C₁-C₂ alkyl.

Also provided is a compound of the Formula (E-1):

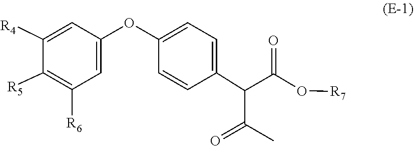

wherein: R₄, R₅, and R₆ are each independently selected from the group of H, F, Cl, Br, CN, —OCH₃, —CH₂F, —CHF₂, —CF₃, —SF₅, and —CH₃; and R₇ is C₁-C₆ alkyl; with the proviso that, when R₇ is methyl, at least one of R₄, R₅, and R₆ is not H.

An additional embodiment provides a compound of Formula (E-1) wherein R₄, R₅, and R₆ are each independently selected from the group of H, F, Cl, Br, CN, —CH₂F, —CHF₂, —CF₃, —OCH₂F, —OCH₃, —OCHF₂, —OCF₃, —SF₅, and —CH₃; and R₇ is C₁-C₅ alkyl; with the proviso that, when R₇ is methyl, at least one of R₄, R₅, and R₆ is not H.

Another embodiment provides a compound of Formula (E-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl; with the proviso that, when R₇ is methyl, at least one of R₄, R₅, and R₆ is not H.

Another embodiment provides a compound of Formula (E-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl; with the proviso that, when R₇ is methyl, at least one of R₄, R₅, and R₆ is not H.

Another embodiment provides a compound of Formula (E-1) wherein R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl; with the proviso that, when R₇ is methyl, at least one of R₄, R₅, and R₆ is not H.

A further embodiment provides a compound of Formula (E-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

A further embodiment provides a compound of Formula (E-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₅ alkyl.

A further embodiment provides a compound of Formula (E-1) wherein R₅ is —OCF₃; R₄, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

A still further embodiment provides a compound of Formula (E-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

A still further embodiment provides a compound of Formula (E-1) wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

Another embodiment provides a compound of Formula (E-1) wherein R₅ is —OCF₃; R₄ and R₆ are each H; and R₇ is C₁-C₆ alkyl.

Another embodiment provides a compound of Formula (E-1) wherein R₅ is —OCF₃; R₄ and R₆ are each H; and R₇ is C₁-C₄ alkyl.

Another embodiment provides a compound of Formula (E-1) wherein R₅ is —OCF₃; R₄ and R₆ are each H; and R₇ is C₁-C₃ alkyl.

Another embodiment provides a compound of Formula (E-1) wherein R₅ is —OCF₃; R₄ and R₆ are each H; and R₇ is C₁-C₂ alkyl.

A further embodiment provides a compound of Formula (G-1):

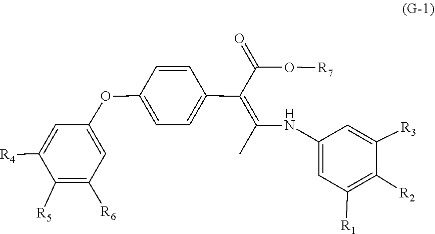

wherein:
R₁ is selected from the group of H and F;
R₂ is selected from the group of H, F, and Cl;
R₃ is selected from the group of H, F, and —OCH₃;
R₄, R₅, and R₆ are each independently selected from the group of H, F, Cl, Br, CN, —OCH₃, —CH₂F, —CHF₂, —CF₃, —SF₅, and —CH₃; and
R₇ is C₁-C₆ alkyl.

An additional embodiment provides a compound of Formula (G-1) wherein R₁ is selected from the group of H and F; R₂ is selected from the group of H, F, and Cl; R₃ is selected from the group of H, F, and —OCH₃; R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₅ alkyl.

An additional embodiment provides a compound of Formula (G-1) wherein R₁ is selected from the group of H and F; R₂ is selected from the group of H, F, and Cl; R₃ is selected from the group of H, F, and —OCH₃; R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

A further embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

A further embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₅ alkyl.

A further embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₄ alkyl.

A still further embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

A still further embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each H; and R₇ is C₁-C₆ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each H; and R₇ is C₁-C₄ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each H; and R₇ is C₁-C₃ alkyl.

Another embodiment provides a compound of Formula (G-1) wherein R₁ is selected from the group of H and F; R₂ is selected from the group of H, F, and Cl; R₃ is selected from the group of H, F, and —OCH₃; R₅ is —OCF₃; R₄ and R₆ are each H; and R₇ is C₁-C₂ alkyl.

Yet another embodiment is a method for the preparation of a compound of Formula (I):

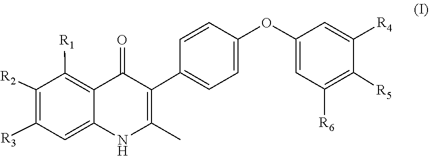

wherein:
R₁ is selected from the group of H, F, Cl, Br, CN, —CH₂F, —CHF₂, —CF₃, —OCH₃, —OCH₂F, —OCHF₂, —OCF₃, —SF₅, and —CH₃;
R₂ is selected from the group of H, F, Cl, Br, CN, —CH₂F, —CHF₂, —CF₃, —OCH₃, —OCH₂F, —OCHF₂, —OCF₃, —SF₅, and —CH₃;
R₃ is selected from the group of H, F, Cl, Br, CN, —CH₂F, —CHF₂, —CF₃, —OCH₃, —OCH₂F, —OCHF₂, —OCF₃, —SF₅, and —CH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃;
   the method involving the steps of:
a) reacting an optionally substituted phenol compound of Formula (A)

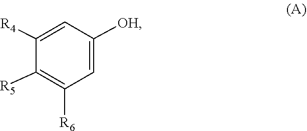

wherein R₄, R₅, and R₆ are as defined above, with 4-bromophenylacetic acid, in the presence of a copper catalyst, to form intermediate diaryl ether carboxylic acid (H);

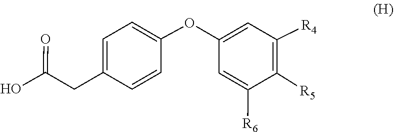

b) removing the copper catalyst, for instance using APDTC;
c) esterifying the diaryl ether carboxylic acid (16) to form an optionally substituted alkyl 2-(4-phenoxyphenyl)acetate compound of Formula (C)

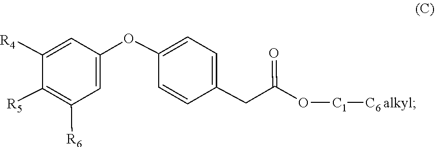

d) treating the compound of Formula (C) with a non-nucleophilic base and acetic anhydride to prepare a compound of Formula (D)

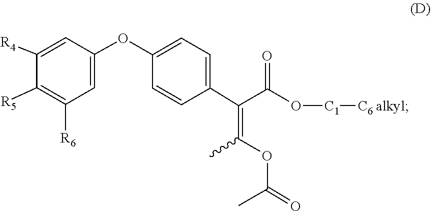

e) treating the compound of Formula (D) with an acid to prepare a compound of Formula (E)

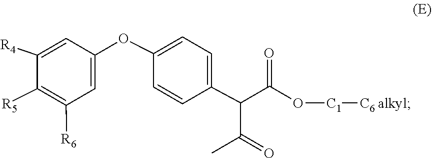

f) reacting the compound of Formula (E) with an optionally substituted aniline compound of Formula (F)

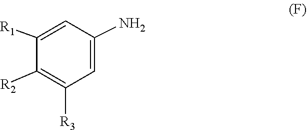

to prepare a compound of Formula (G)

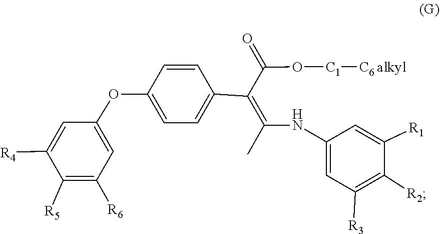

and
g) heating the compound of Formula (G) at a temperature of from about 150° C. to about 300° C. to prepare the compound of Formula (I).

Also provided is an embodiment that is a method of preparing a compound of Formula (C):

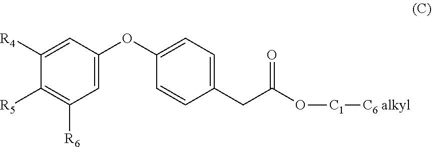

the method involving:
reacting, in the presence of a copper catalyst, 4-bromophenylacetic acid (11a) with a phenol moiety substituted by R₄, R₅, and R6, as defined herein, to form the intermediate diaryl ether carboxylic acid (H);
removing the copper catalyst; and
esterifying the carboxylic acid (H) to Compound (C).

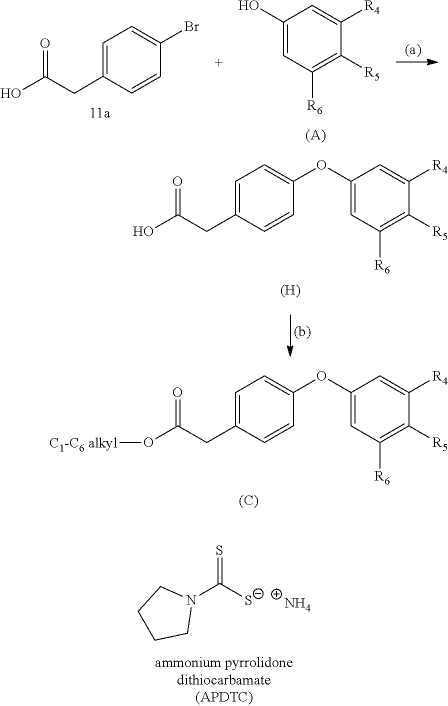

Yet another method embodiment is a method of preparing Compound (C), involving: reacting 4-bromophenylacetic acid (11a) with a phenol (A) substituted by R₄, R₅, and R₆, as defined herein, in a first medium involving a copper catalyst to form a second medium involving the intermediate diaryl ether carboxylic acid (H) and the copper catalyst;
c) removing the copper catalyst from the second medium with a copper chelating agent to create a third medium involving the intermediate diaryl ether carboxylic acid (H); and
d) reacting the intermediate diaryl ether carboxylic acid (H) in the third medium with a C₁-C₆ alkanol (for instance, ethanol) to form esterified Compound (C).

In examples of this method, the copper chelating agent is or includes a dithiocarbamate chelating agent, such as ammonium pyrrolidine dithiocarbamate (APDTC), sodium dimethyldithiocarbamate (NaDMDTC), or sodium diethyldithiocarbamate (NaDEDTC).

In certain specific examples, the copper chelating agent is ammonium pyrrolidine dithiocarbamate (APDTC).

Also provided are methods of synthesis of ethyl 2-(4-(4-(trifluoromethoxy)phenoxy)phenyl)acetate 12, which methods do not involve high vacuum distillation.

Yet another embodiment is a method of preparing a compound of Formula (I):

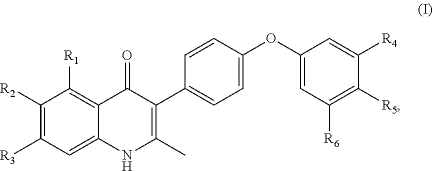

wherein: R₁, R₂, R 3 , R₄, R₅ and R₆ are each independently selected from among H, F, Cl, Br, CN, —CH₂F, —CHF₂, —CF₃, —OCH₃, —OCH₂F, —OCHF₂, —OCF₃, —SF₅, and —CH₃; and which method does not involve or require high vacuum distillation.

### Detailed Description

In an embodiment, Compound (C) may be prepared by reacting, in the presence of a copper catalyst, 4-bromophenylacetic acid (11a) with a phenol moiety (A) substituted by R₄, R₅, and R₆, as defined herein, to form the intermediate diaryl ether carboxylic acid (H). The copper catalyst may then be removed using APDTC and the carboxylic acid may be esterified to Compound (C).

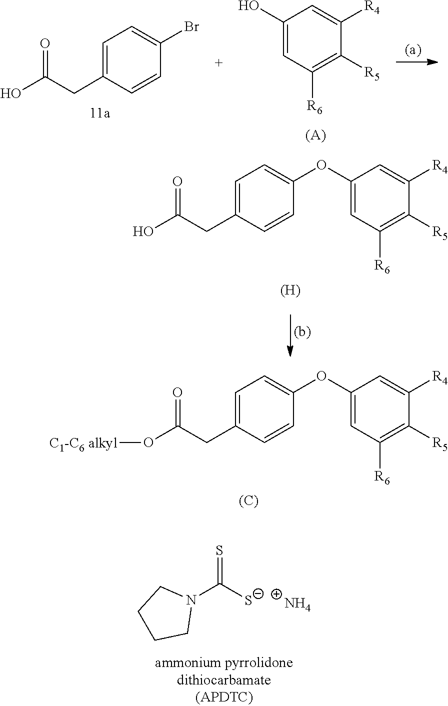

Another embodiment provides a process of preparing Compound (C),

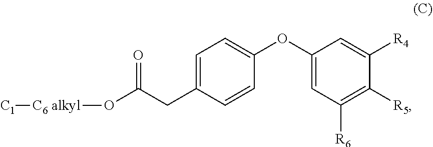

the process comprising the steps of:
reacting 4-bromophenylacetic acid (11a) with a phenol (A) substituted by R₄, R₅, and R₆ (A), as defined herein, in a first medium comprising a copper catalyst to form a second medium comprising the intermediate diaryl ether carboxylic acid (H) and the copper catalyst;
removing the copper catalyst from the second medium with a copper chelating agent to create a third medium comprising the intermediate diaryl ether carboxylic acid (H); and
reacting the intermediate diaryl ether carboxylic acid (H) in the third medium with a c₁-c₆ alkanol to form esterified Compound (C).

Non-limiting ligands useful in step a) include N,N-dimethylglycine, 3-(dimethylamino)propanoic acid, 2-(pyridin-2-yl)acetic acid, 8-((di(furan-2-yl)phosphaneyl)oxy)quinoline, 8-((dicyclohexylphosphaneyl)oxy) quinoline, and (1E,1′E)-N,N′-(ethane-1,2-diyl)bis(1-(thiophen-2-yl)methanimine). Additional ligands for use in the methods herein may be found in, Otto et al. (Beilstein J. Org. Chem. 8:1105-1111, 2012).

In some embodiments, the copper chelating agent in step b), above, is a dithiocarbamate chelating agent, such as ammonium pyrrolidine dithiocarbamate (APDTC), sodium dimethyldithiocarbamate (NaDMDTC), or sodium diethyldithiocarbamate (NaDEDTC). In some embodiments, the copper chelating agent is ammonium pyrrolidine dithiocarbamate (APDTC).

In some embodiments, the C₁-C₆ alkanol is ethanol.

Another provided embodiment described herein is a modified procedure, in which the synthesis of the diaryl ether 12 does not involve high vacuum distillation. This synthesis is illustrated in Scheme 3b. Using the Ullmann coupling, reaction of 4-bromophenylacetic acid 11a with 4-trifluoromethoxyphenol 3 in the presence of catalytic amount of copper (I) chloride (CuCl), N,N,-dimethylglycine (DMG), and potassium carbonate (K₂CO₃) in dimethylformamide (DMF), for example at 160° C. for 3.5 hours, gives solid carboxylic acid diaryl ether 14 (2-(4-(4-(trifluoromethoxy)phenoxy)phenyl)acetic acid) after recrystallization, for example from hexane/ethylacetate. The copper catalyst can be removed by adding a copper chelator, for example ammonium pyrrolidinedithiocarbamate (APDTC), during reaction workup according to Gallagher & Vo (Org Proc Res & Dev., 19(10):1369-1373, 2014). Esterification of 14 with ethanol in the presence catalytic hydrochloric acid affords the desired diaryl ether 12.

The product of this reaction was then reacted to form the desired β-keto-ethylester diarylether intermediate that is central to the large-scale production of ELQ-300, ELQ-316, as well as other high-value ELQs with potential for use in prevention, treatment and cure of parasitic infections of humans and animals. A key advantage is that this new reaction sequence avoids the use of high vacuum distillation in the synthesis of the key beta-keto-ester intermediate, thus providing a safe low cost and high yield alternative synthetic route to this valuable intermediate. High temperature vacuum distillation can give rise to both reactive and decomposition hazards in process scale reactions.

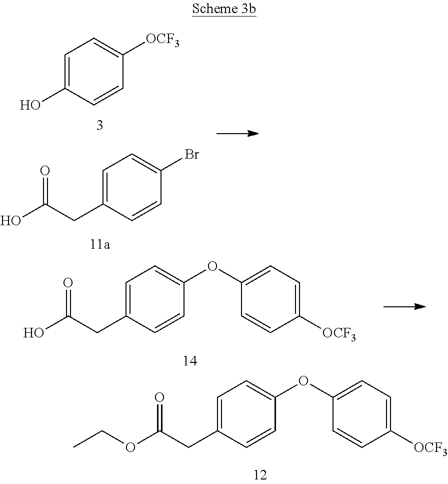 [SMILES: Oc1ccc(OC(F)(F)F)cc1 ; O=C(O)Cc1ccc(Br)cc1 ; O=C(O)Cc1ccc(Oc2ccc(OC(F)(F)F)cc2)cc1 ; CCOC(=O)Cc1ccc(Oc2ccc(OC(F)(F)F)cc2)cc1]

The acid 14 illustrated at the center (above) is novel. A description of its synthesis, purification, removal of copper, and conversion to the desired β-keto ester 12, ethyl 2-(4-(4-(trifluoromethoxy)phenoxy)phenyl)acetate, is described in Example 2.

### Definitions

All ranges disclosed and/or claimed herein are inclusive of the recited endpoint and independently combinable. For example, the ranges of “from 2 to 10” and “2-10” are inclusive of the endpoints, 2 and 10, and all the intermediate values between in context of the units considered. For instance, reference to “claims 2-10” or “C₂-C₁₀ alkyl” includes units 2, 3, 4, 5, 6, 7, 8, 9, and 10, as claims and atoms are numbered in sequential whole numbers without fractions or decimal points, unless described in the context of an average number. The context of “pH of from 5-9” or “a temperature of from 5° C. to 9° C.”, on the other hand, includes whole numbers 5, 6, 7, 8, and 9, as well as all fractional or decimal units in between, such as 6.5 and 8.24.

The term “independently selected” refers to a situation when more than one variable or item may be selected from a list of options regardless of which of the options applies to another variable or item. For instance, when variables R₁ and R₂ may be independently selected from the group of A, B, C, and D, R₁ and R₂ may each comprise the same option from the list (i.e., R₁ is A and R₂ is A) or different options from the list (i.e., R₁ is A and R₂ is C).

The term “alkyl” refers to a straight or branched hydrocarbon. For example, an alkyl group can have 1 to 6 carbon atoms (i.e., C₁-C₆ alkyl or C₁₋₆ alkyl), 1 to 4 carbon atoms (i.e., C₁-C₄ alkyl or C₁₋₄ alkyl), 1 to 3 carbon atoms (i.e., C₁-C₃ alkyl or C₁₋₃ alkyl), or 1 to 2 carbon atoms (i.e., C₁-C₂ alkyl or C₁₋₂ alkyl). Examples of suitable alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

A “heat transfer fluid” is a liquid or a gas that transports heat from one component to another component in any process requiring heating or cooling, or simply to maintain a constant or relatively constant temperature. A heat transfer fluid used in the methods and steps herein may be a non-reactive organic solvent with a boiling point above the temperature at which a given method or step is to occur. Examples of such heat transfer agents or high boiling point organic solvents include, but are not limited to, DOWTHERM A™ heat transfer fluid (diphenyl plus diphenyl ether), diphenyl ether, diphenyl, biphenyl, methyl benzoate, ethyl benzoate, propyl benzoate, isopropyl benzoate, butyl benzoate, isopentyl (isoamyl) benzoate, phenyl benzoate, benzyl benzoate, isomers of dibenzyitoluene, partially hydrogenated terphenyls, diphenyl methane, mineral oil, and the like.

As used herein, the term “flow reactor”, also known as a continuous reactor, refers to a reactor, sometimes of cylindrical geometry, that allow chemical reactions to occur in a continuous, flowing system, such as a plug flow reactor.

It is understood that the generic and specific structures and chemical names for individual compounds, as final products or intermediate compounds, include the compound's stereoisomer, mixture of stereoisomers, or tautomer thereof. For instance, it is understood that each of the two tautomeric structures below includes, encompasses, and represents the compound of the other.

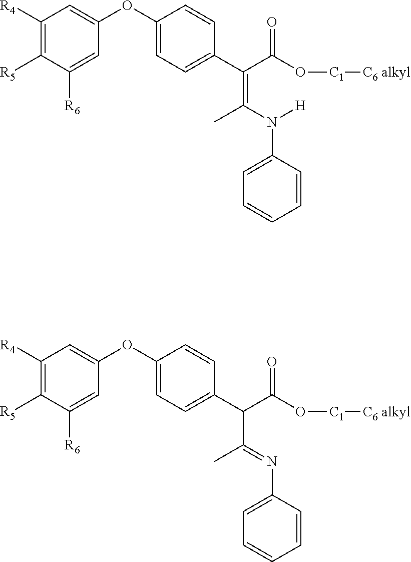

### Example 1: Synthetic Route to Produce ELQ-300 and Structurally Related ELQ Derivatives

Synthesis of β-keto ester 15 begins with the synthesis of diaryl ether 12 (ethyl 2-(4-(4-(trifluoromethoxy)phenoxy)phenyl)acetate) using an Ullmann reaction (Fui et al., Catalysts 2020, 10 (10)). The copper-mediated coupling of 4-trifluoromethoxyphenol 3 and ethyl-2-(4-bromophenyl)acetate 11b with copper (I) chloride (CuCl), N,N-dimethylglycine (DMG) as copper (I) chloride chelator and potassium carbonate (K₂CO₃) in dimethylformamide (DMF) at 160° C. for 120 minutes afforded the diaryl ether 12 via the Ullmann reaction (Scheme 3; Ma & Cai, Org Lett 2003, 5 (21), 3799-802; Kancharla et al., J Med Chem 2020, 63 (11), 6179-6202).

The reaction was monitored by GC-MS and was determined to be complete when 11b was consumed. The product 12 was isolated with a yield of 48-60% using high vacuum distillation, a method that facilitated the nearly complete removal of copper. The copper content of 12 using these conditions was <21 ppm, determined using Inductively Coupled Plasma-Atomic Emission Spectroscopy (ICP-AES). Other copper couplings were explored, but they required harsh conditions (e.g., copper metal) or were not scalable (e.g., copper (II) acetate; Tekale et al., Mini-Reviews in Org Chem., 10(3): 281-301, 2013). The copper (II) acetate reaction (not shown) used in the original ELQ synthetic method (Nilsen et al., Sci Transl Med 2013, 5(177), 177ra37; Nilsen et al., J Med Chem 2014, 57 (9), 3818-34) did not scale well because it was difficult to maintain a dry, well-oxygenated, homogenous copper (II) acetate mixture under bulk conditions.

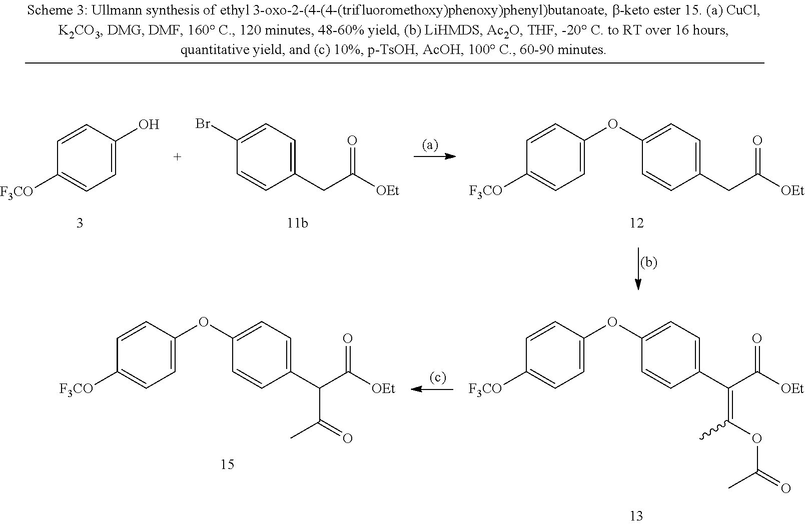

With the diaryl ether 12 in hand, a challenging part of this route, i.e., the formation of the substituted β-keto ester 15, was explored. The acylation (reaction b, Scheme 3) did not proceed using strong bases such as NaH, n-butyllithium, lithium diisopropylamide, and commercially prepared lithium hexamethyldisilazide (LiHMDS) solution. We found that the acylation proceeded only when freshly prepared LiHMDS was used to deprotonate 12 at −20° C. Preliminary attempts to acylate 12 suggested that C-acylation occurs initially, followed by rapid O-acylation of the newly-introduced acetyl group, producing enol acetate 13. If the reaction is quenched at −20° C. immediately after addition of acetic anhydride, both C-acylated 15, the bis-acylated 13, and the starting material 12 can be detected by thin layer chromatography (TLC). This finding suggested that it was necessary to use excess acetic anhydride with freshly prepared LiHMDS in tetrahydrofuran (THF) to force the reaction to consume all of the starting material 12, thereby forming only the O-acylated β-keto ester 13. Under these conditions, 13 was obtained in quantitative yield and with sufficient purity (>95%) to be used in the next step without further purification. Compound 13 exists as a mixture of equally-reactive E- and Z-isomers, which can be isolated using flash chromatography. The identity of the Z-isomer was determined by 2-D Nuclear Overhauser Effect Spectroscopy (NOESY) NMR. The percent Z-isomer was estimated to be 90-95% using GC-MS and ¹H-NMR.

The O-acylated β-keto ester 13 can be quantitatively converted to the desired β-keto ester-15 using catalytic para-toluenesulfonic acid (p-TsOH) in acetic acid (AcOH). The β-keto ester-15 exists in both keto and enol forms with a keto/enol ratio of approximately 70:30 as determined by ¹H-NMR. Since the β-keto ester 15 cannot be detected by GC-MS, TLC and ¹H-NMR was used to characterize the product and to assess purity. The isolated β-keto ester 15 was sufficiently pure for use in the next reaction and already contained a 10% mole fraction of p-TsOH, which is a suitable catalyst for the subsequent acid-catalyzed aniline condensation.

The target ELQ compounds were prepared from β-keto ester 15 using a Conrad-Limpach reaction, which comprises a Schiff base formation followed by a high-temperature cyclization (Scheme 6). Continuous removal of water using a Dean-Stark trap and a water-carrying solvent affords the desired Schiff base via condensation with anilines 19a-d. Traditionally, benzene was used (Salzer et al., Chemische Berichte 1948, 81 (1), 12-19; Winter et al., Exp Parasitol 2011, 127 (2), 545-51; Winter et al., Exp Parasitol 2008, 118 (4), 487-97); however, since benzene is not preferred for pharmaceutical preparations, cyclohexane (which boils at nearly the same temperature and also forms an azeotrope with water) was identified as an alternative. Anilines 19a-d were allowed to react with the isolated β-keto ester 15 in the presence of catalytic p-TsOH in refluxing cyclohexane with a Dean-Stark trap to give the imines 20a-d, which were then used without further purification in the final cyclization step.

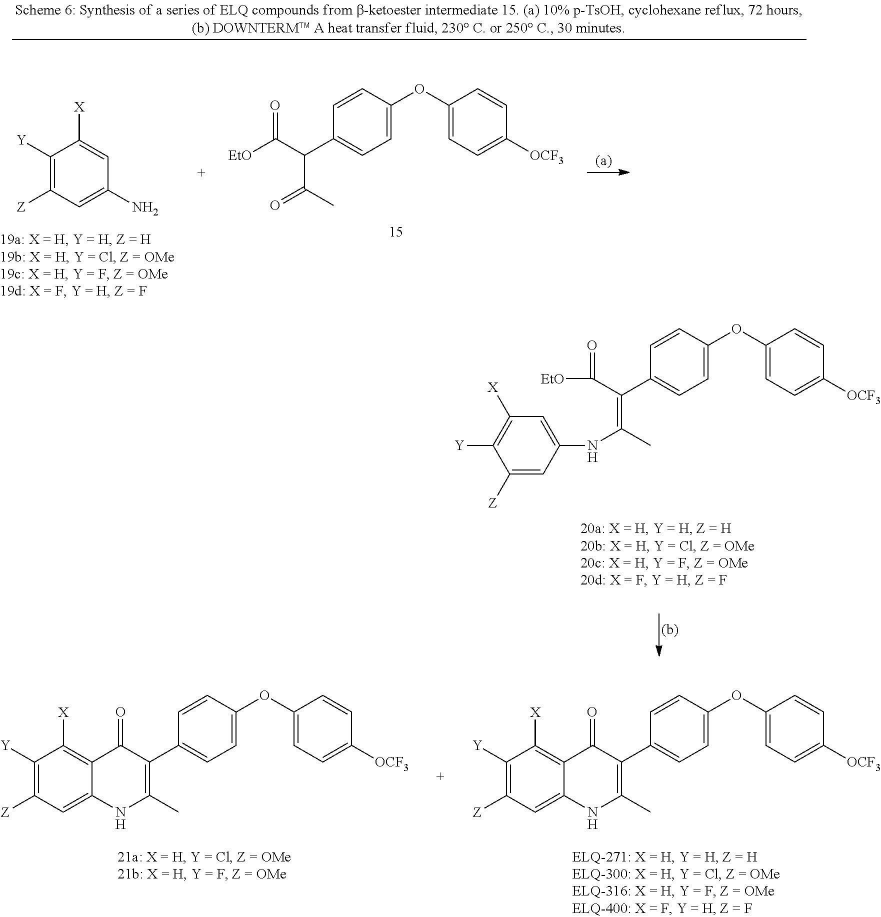

The cyclization step of the Conrad-Limpach reaction is classically performed at high temperatures (250° C. or 230° C.) (Elderfield, Heterocyclic Compounds. John Wiley & Sons, Inc., New York, NY, 1961). These two temperatures were compared for a series of ELQ derivatives and investigated the effect of a lower temperature of 200° C. for ELQ-300 (Table 1). All temperatures investigated led to acceptable yields of product with minimal impurity, however, reaction at 200° C. led to a somewhat lower yield of ELQ-300. Although aniline 19b was used as the limiting reagent, it was not completely consumed even after extending the reaction time to 72 hours. Excess aniline 19b, being poorly soluble in cyclohexane, was partially recovered by filtration.

To demonstrate the potential scalability of this synthetic route, the reaction was performed using 52.25 g of β-keto ester 15 and 2.25 g of p-TsOH to form the Schiff base 20b followed by cyclization at 250° C. Under these conditions, the reaction proceeded as expected and gave ELQ-300 in a yield of 57.7%, higher than at the 1-10 g (2-20 mmol) scale. The crude product from this reaction was >99% pure by ¹H-NMR and HPLC. At all temperatures investigated, only a negligible amount of regioisomer 21a of ELQ-300 was formed, which was below the limit of quantification (not detected) by ¹H-NMR. To increase the limit of quantification of 21a, the product was converted to its corresponding 4-chloro derivative using phosphorus oxychloride (POCl₃) and analyzed by GC-MS. The results confirmed that a negligible amount (<0.5%) of the regioisomer 21a was formed. In this study, when the reactions were performed at 1-10 (2-20 mmol) g scale, 230° C. appeared to be the optimal cyclization temperature as it gave the highest yield. However, a higher yield was obtained when the reaction was scaled up to 52.25 g of β-keto ester 15 at 250° C. Other solvents may work in this reaction (Brouet et al., Synth Commun 2009, 39 (9), 5193-5196). Similar reactions requiring such temperatures have been performed using a flow reactor, which demonstrates that this approach should be amenable to optimization on an industrial scale (Tsoung et al., J Org Chem 2017, 82 (2), 1073-1084; Bogdan et al., J Med Chem. 2019, 62 (14), 6422-6468).

Next, the general utility of using the β-keto ester 15 as an intermediate for the synthesis of other ELQ compounds was investigated, including ELQ-271, ELQ-316, and ELQ-400 (Nilsen et al., Sci Transl Med 2013, 5 (177), 177ra37; Nilsen et al., J Med Chem 2014, 57(9), 3818-34; Stickles et al., Am J Trop Med Hyg 2015, 92 (6), 1195-201). In all cases, the condensation of β-keto ester 15 with the corresponding aniline 19a, 19b, 19c, and 19d, respectively, followed by cyclization, afforded the desired quinolones in relatively good yield and with minimal formation of undesired regioisomers as detected by GC-MS of the corresponding 4-chloro derivatives (Scheme 6).

### Conclusion

Provided in this Example is an efficient late-stage cyclization synthetic route to ELQ-300 and other structurally related ELQ derivatives, bypassing the more expensive palladium catalyzed Suzuki-Miyaura reaction originally developed by our lab to explore the initial structure-activity relationship of quinolone and side chain modifications to the core scaffold (Nilsen et al., Sci Transl Med 2013, 5 (177), 177ra3; Nilsen et al., J Med Chem 2014, 57 (9), 3818-34). The key to the success of this efficient late-stage cyclization scheme is the ability to synthesize the bis-acylated product 13 and then to selectively convert it to the desired intermediate β-keto ester 15 using p-TsOH as a catalyst. This synthetic route is inexpensive and contains four scalable steps, with one distillation, one recrystallization (as needed) and no chromatography. ELQ-300 and other ELQ compounds are obtained in pure form with an overall yield of 25-30%. Further, the steps have not been optimized, and it is believed that improvements may be achievable by adjustments in reaction conditions (e.g., time and temperature), solvents, and equipment. Compared to the synthetic route for ELQ-300 and other ELQ compounds originally described by us (Nilsen et al., Sci Transl Med 2013, 5 (177), 177ra3; Nilsen et al., J Med Chem 2014, 57(9), 3818-34), this late-stage cyclization synthesis appears to be readily scalable and promises to considerably reduce the cost of goods.

In addition to the advantages described above, this efficient synthetic route avoids the relatively harsh conditions (HBr in acetic acid) required to deprotect the 4-O-ethyl ether quinoline intermediate in the previously described synthesis (Nilsen et al., Sci Transl Med 2013, 5 (177), 177ra3; Nilsen et al., J Med Chem 2014, 57 (9), 3818-34). These conditions regularly resulted in the cleavage of the 7-OMe ether of both ELQ-300 and ELQ-316.

It is also noteworthy that ELQ-316 can also be made efficiently and with high purity and yield by this new late-stage cyclization synthetic pathway. While ELQ-316 exhibits slightly less antimalarial activity than ELQ-300 in vivo, it has superior antiparasitic activity against a broader range of apicomplexan protozoan species including Toxoplasma gondii and Babesia microti which also cause potentially fatal disease in humans. Recent studies also show that both ELQ-300 and ELQ-316 are active against a range of apicomplexan parasites of importance to veterinary medicine (Silva et al., Parasit Vectors 2020, 13 (1), 606; Eberhard et al., Front Vet Sci 2020, 7, 96; Anghel et al., Front Vet Sci 2018, 5, 285).

### Example 2: Alternative Synthetic Route to Produce ELQ-300 and Structurally Related ELQ Derivatives

A second optimized approach to produce 3-diaryl ether 4(1H)-quinolones, presented in this Example, is also relatively short (5 reaction steps), does not require palladium, involves no chromatographic separation, and requires no protecting group chemistry because the poorly soluble 4(1H)-quinolone is not formed until the final reaction step. This approach also avoids high vacuum distillation. Material in this Example overlaps with work described in Example 1; at least some of the experimental results provided herein were also published in Pou et al., Org. Process Res. Dev 25:1841-1852, 2021; ePub August 4, 2021 (doi: 10.1021/acs.oprd.1c00099).

Synthesis of β-keto ester 15 begins with the synthesis of diaryl ether 12 using an Ullmann reaction (Fui et al., Catalysts 2020, 10(10)). The copper-mediated coupling of 4-trifluoromethoxyphenol 3 and ethyl-2-(4-bromophenyl)acetate with copper (I) chloride (CuCl), N,N-dimethylglycine (DMG) as copper (I) chloride chelator and potassium carbonate (K₂CO₃) in DMF at 160° C. for 90 min afforded the diaryl ether 12 via the Ullmann reaction (Scheme 3) (Ma & Cai, Org Lett 2003, 5 (21), 3799-802; Kancharla et al., J Med Chem 2020, 63 (11), 6179-6202). The reaction was monitored by GC-MS and was determined to be complete when 11 was consumed. The product 12 was isolated with a yield of 48-60% using high vacuum distillation, a method that facilitated the nearly complete removal of copper. The copper content of 12 using these conditions was <21 ppm, determined using Inductively Coupled Plasma-Atomic Emission Spectroscopy (ICP-AES). Other copper couplings were explored, but they required harsh conditions (e.g., copper metal) or were not scalable (e.g., copper (II) acetate) (Sunil, et al., Mini-Reviews in Org Chem 2013, 10 (3), 281-301). The copper (II) acetate reaction (not shown) used in the original ELQ synthetic method (Nilsen et al., Sci Transl Med 2013, 5 (177), 177ra37; Nilsen et al., J Med Chem 2014, 57 (9), 3818-34) did not scale well because it was difficult to maintain a dry, well-oxygenated, homogenous copper (II) acetate mixture under bulk conditions.

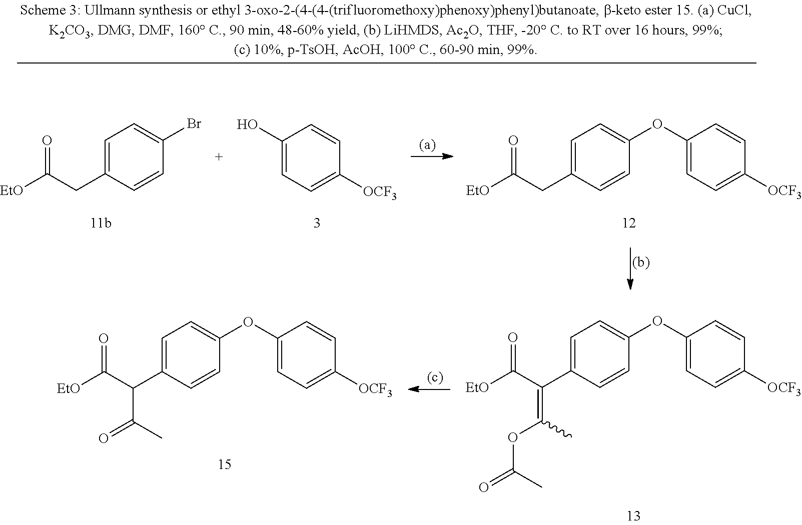

Two alternate routes can be seen below for the Ullmann synthesis of ethyl 2-(4-(4-(trifluoromethoxy)phenoxy)phenyl)acetate 12. (a) CuCl, K₂CO₃, DMG, DMF, 160° C., 90 min, 48-60% yield; (b) 1. CuCl, K₂CO₃, DMG, DMF, 160 ° C., 3.5 hours, and 2. APDTC, 78%; (c) catalytic HCl, EtOH, 18 hours, 91%.

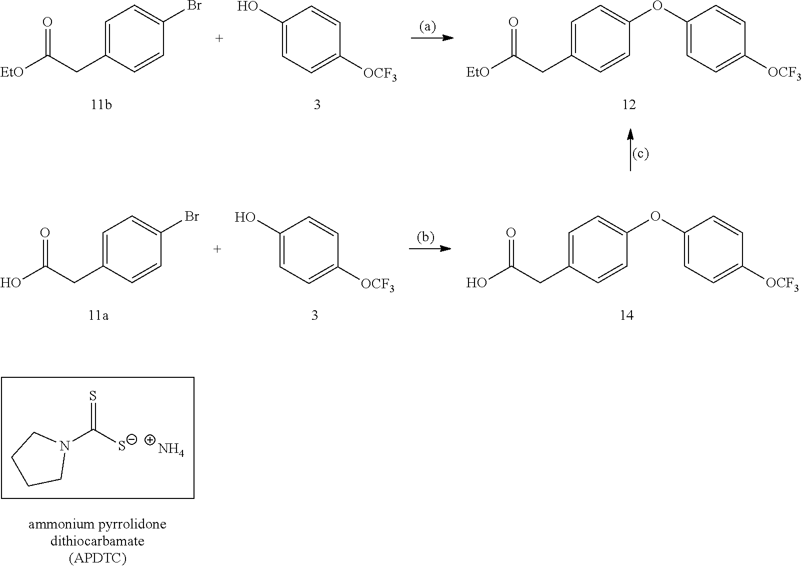

In order to avoid high vacuum distillation, which may be problematic on an industrial scale, we attempted to find an alternative method for the preparation of diaryl ether 12 (ethyl 2-(4-(4-(trifluoromethoxy)phenoxy)phenyl)acetate). By substituting the corresponding carboxylic acid for the ester in the Ullmann reaction, we obtained a solid intermediate, diaryl ether 14, that could be purified without distillation (Scheme 4, reaction a). 4-Bromophenylacetic acid 11a was allowed to react with 4-trifluoromethoxyphenol 3 in the presence of a catalytic amount of CuCl, DMG, and K₂CO₃ in DMF at 160° C. After 3.5 hours 11a was completely consumed as determined by GC-MS. During the workup, the copper catalyst was removed by addition of ammonium pyrrolidinedithiocarbamate (APDTC) according to the procedure described by Gallagher and Vo (Org Proc Res & Dev 2014, 19 (10), 1369-1373). The solid carboxylic acid 14 was easily purified by washing with boiling water to remove most of the primary impurity (starting material, phenol 3) and water-soluble residues, and was obtained in 78% yield with >95% purity by GC-MS and ¹H-NMR. Esterification of 14 with ethanol in the presence of catalytic hydrochloric acid over 18 hours afforded the desired diaryl ether 12 in 91% yield after removal of ethanol in vacuo and passage of the crude product through a silica gel plug, which was rinsed with 3:1 hexanes/ethyl acetate. The product 12 was at least 95% pure by GC-MS and ¹H-NMR and was suitable for use in the next step without further purification. The overall yield of this two-step process was 71%. The successful preparation of the solid intermediate 14 and its conversion to the high purity key intermediate 12 shows that this ELQ compound synthesis can be performed without high vacuum distillation.

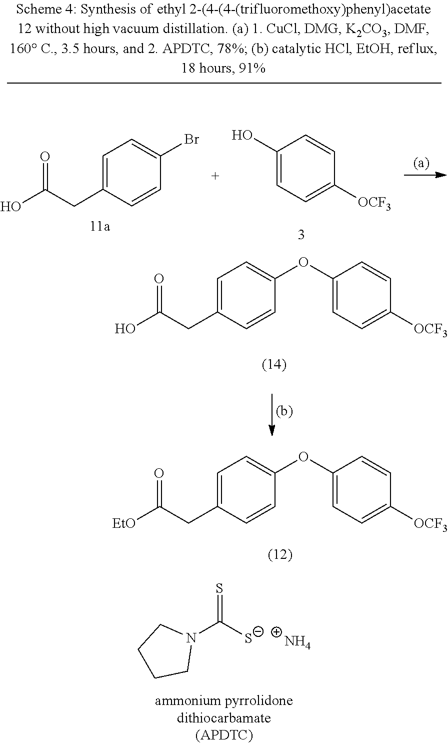

It was important to verify that the copper content of 12 obtained via the carboxylic acid route (Scheme 4) was as low as the copper content of 12 obtained via the ester route (Scheme 3). Using ICP-AES analysis, it was determined that the copper content of 14 after treatment with APDTC was 73 ppm. After esterification and passage through a silica gel plug, the copper content of 12 was <12 ppm. Thus, the copper content of 12 obtained according to Scheme 4 was no higher than the copper content of 12 (<21 ppm) obtained after high vacuum distillation (Scheme 3).

With diaryl ether 12 in hand, the most challenging part of this route, the formation of the key intermediate, the substituted β-keto ester 15, was explored.

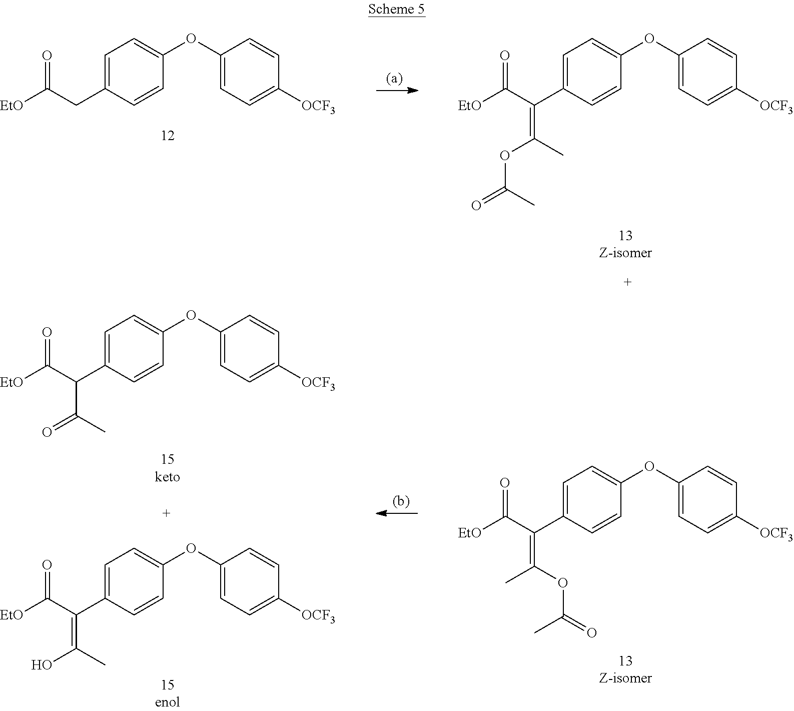 [SMILES: CCOC(=O)Cc1ccc(Oc2ccc(OC(F)(F)F)cc2)cc1 ; CCOC(=O)/C(=C(/C)OC(C)=O)c1ccc(Oc2ccc(OC(F)(F)F)cc2)cc1 ; CCOC(=O)C(C(C)=O)c1ccc(Oc2ccc(OC(F)(F)F)cc2)cc1 ; CCOC(=O)/C(=C(/C)O)c1ccc(Oc2ccc(OC(F)(F)F)cc2)cc1 ; CCOC(=O)/C(=C(\C)OC(C)=O)c1ccc(Oc2ccc(OC(F)(F)F)cc2)cc1]

The acylation (Scheme 5, above, and Scheme 3, reaction b) did not proceed using strong bases such as NaH, n-butyllithium, lithium diisopropylamide, and commercially prepared lithium hexamethyldisilazide (LiHMDS) solution. It was found that the acylation proceeded only when freshly prepared LiHMDS was used to deprotonate 12 at −20° C. Preliminary attempts to acylate 12 suggested that C-acylation occurs initially, followed by rapid O-acylation of the newly introduced acetyl group, producing enol acetate 13. If the reaction is quenched at −20° C. immediately after addition of acetic anhydride, C-acylated 15, bis-acylated 13 and the starting material 12 can be detected by thin layer chromatography (TLC). This finding suggested that it was necessary to use excess acetic anhydride with freshly prepared LiHMDS in tetrahydrofuran (THF) to force the reaction to consume all of the starting material 12, thereby forming only the O-acylated β-keto ester 13. Under these conditions, 13 was obtained in quantitative yield and of sufficient purity (>95%) to be used in the next step without further purification. Compound 13 exists as a mixture of equally-reactive E- and Z-isomers, which can be isolated using chromatography. The identity of the Z-isomer was determined by 2-D NOESY NMR, and the percent Z-isomer was estimated to be 90-95% using GC-MS and ¹H-NMR.

Other acylating reagents were explored. Ethyl acetate and acetyl-imidazole have been reported to give 2-phenylacetoacetic esters in one step (Vaswani et al., Org Lett 2014, 16 (16), 4114-7), and, indeed, our model reactions using ethyl 2-(4-bromophenyl)acetate provided direct access to the corresponding β-keto ester. Unfortunately, reactions with diaryl ether 12 proved to be more complex. In the case of ethyl acetate, we observed no reaction. In the case of acetyl imidazole, β-keto ester 15 was formed predominantly along with some of the bis-acylated product 13 and some other uncharacterized products as shown by ¹H-NMR. Under these conditions, in order to obtain β-keto ester 15 in sufficient purity for the next reaction, chromatography would be required. Thus, in our case, we found that acylation with acetyl imidazole is less desirable and may not be suitable for large scale synthesis, especially considering the relative ease of work-up and low cost associated with acetic anhydride.

We found that it is possible to quantitatively convert the bis-acylated 15 to the desired β-keto ester 15 using catalytic para-toluenesulfonic acid (p-TsOH) in AcOH. The β-keto ester 15 exists in both keto and enol forms with a keto/enol ratio of approximately 7:3 as determined by ¹H-NMR. Since the β-keto ester 15 could not be detected by GC-MS, we used TLC and ¹H-NMR to assess purity and characterize the product. The isolated β-keto ester 15 was sufficiently pure for use in the next reaction and already contained a 10% mole fraction of p-TsOH, which is a suitable catalyst for the subsequent acid-catalyzed aniline condensation.

The target ELQ compounds are prepared from β-keto ester 15 using a Conrad-Limpach reaction, which comprises a Schiff base formation followed by high-temperature cyclization (Scheme 6) (Conrad & Limpach, Berichte der deutschen chemischen Gesellschaft 1887, 20 (1), 944-948; Conrad & Limpach, Berichte der deutschen chemischen Gesellschaft 1891, 24 (2), 2990-2992). Continuous removal of water using a Dean-Stark trap and a water-carrying solvent affords the desired Schiff bases 20a-d via condensation with anilines 19a-d. Traditionally, benzene was used (Salzer et al., Chemische Berichte 1948, 81 (1), 12-19; Winter et al., Exp Parasitol 2011, 127 (2), 545-51; Winter et al., Exp Parasitol 2008, 118 (4), 487-97). However, since benzene is not suitable for pharmaceutical preparations, cyclohexane (which boils at nearly the same temperature and also forms an azeotrope with water) was identified as an alternative. Anilines 19a-d were allowed to react with β-keto ester 15 in the presence of catalytic p-TsOH in refluxing cyclohexane with a Dean-Stark trap to give the imines 20a-d, which were then used without further purification in the final cyclization step.

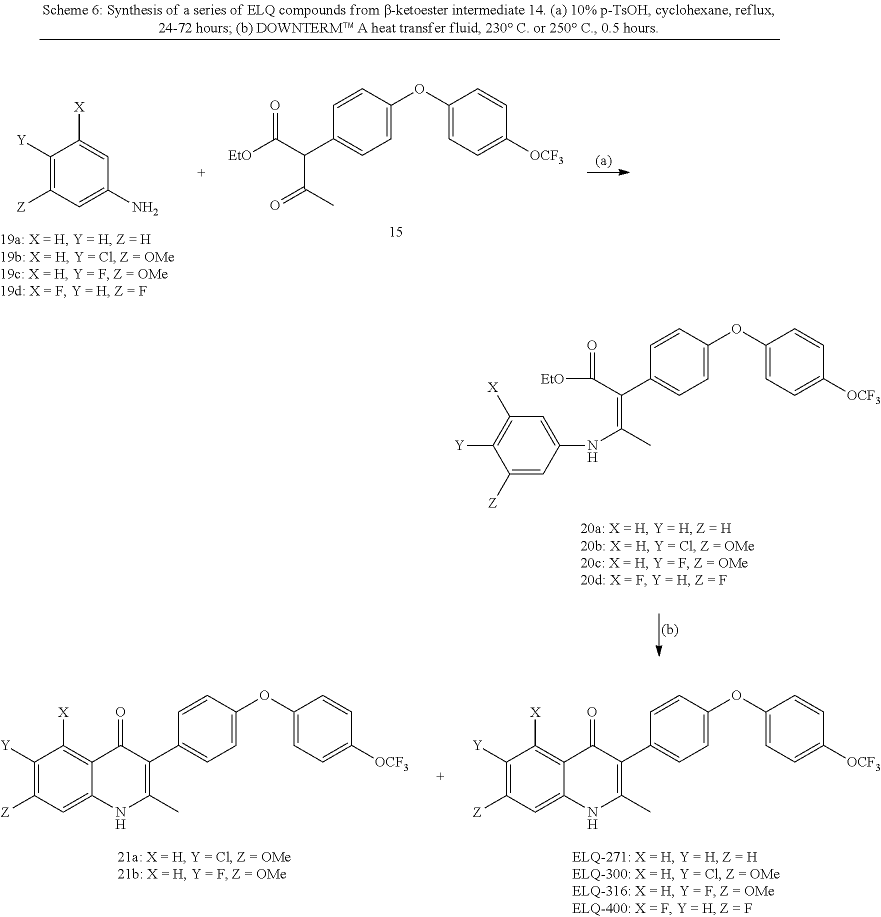

The cyclization step of the Conrad-Limpach reaction is classically performed at high temperatures (230° C. or 250° C.) (Elderfield, Heterocyclic Compounds. John Wiley & Sons, Inc.: New York, NY, 1961). We compared these two temperatures for a series of ELQ derivatives and investigated the effect of a lower temperature of 200° C. for ELQ-300 (Table 1). All temperatures investigated led to acceptable yields of product with minimal impurity, however, reaction at 200° C. led to a somewhat lower yield of ELQ-300. Although aniline 19b was used as the limiting reagent, it was not completely consumed even after extending the reaction time to 72 hours. Excess aniline 19b, being poorly soluble in cyclohexane, was partially recovered by filtration. To demonstrate the potential scalability of this synthetic route, we performed the reaction using 50.0 g (131 mmol) of β-keto ester 15 and 2.25 g of p-TsOH to form the Schiff base 20b followed by cyclization at 250° C. Under these conditions, the reaction proceeded as expected and gave ELQ-300 in a yield of 57.1%, higher than at the 1-10 g (2-20 mmol) scale. The crude product from this reaction was >99% pure by ¹H-NMR and HPLC. At all temperatures investigated, only a negligible amount of regioisomer 21a of ELQ-300 was formed, which was below the limit of quantification (not detected) by ¹H-NMR. To increase the limit of quantification of 21a the product was converted to its corresponding 4-chloro derivative using phosphorus oxychloride (POCl₃) and analyzed by GC-MS. The results confirmed that a negligible amount (<0.5%) of regioisomer 21a was formed. In this study, when the reactions were performed at a 1-10 g (2-20 mmol) scale, 230° C. appeared to be the optimal cyclization temperature as it gave the highest yield. However, a higher yield was obtained when the reaction was scaled up to 50.0 g (131 mmol) of β-keto ester 15 at 250° C. Other solvents may work in this reaction (Brouet et al., Synth Commun 2009, 39 (9), 5193-5196). Tsoung et al. have shown that 4(1H)-quinolones can be prepared by performing a Conrad-Limpach reaction in a flow reactor, which demonstrates that this approach should be amenable to optimization on an industrial scale (Tsoung et al., J Org Chem 2017, 82 (2), 1073-1084; Bogdan et al., J Med Chem 2019, 62 (14), 6422-6468).

TABLE 1
Conrad-Limpach reaction conditions for the
synthesis of a series of ELQ compounds.

| | Amount | | Cyclization | | |
|---|---|---|---|---|---|
| ELQ # | of 15 | | Temperature | Yield¹ | Purity² |
| ELQ-300 | 131 | mmol | 250° C. | 57% | >99% |
| ELQ-300 | 21.5 | mmol⁴ | 250° C. | 43% | >99%³ |
| ELQ-300 | 8.3 | mmol⁴ | 230° C. | 48% | >99% |
| ELQ-300 | 1.8 | mmol⁴ | 200° C. | 30% | >99% |
| ELQ-316 | 302 | mmol | 250° C. | 54% | >99% |
| ELQ-316 | 13.1 | mmol⁴ | 250° C. | 53% | 97% |
| ELQ-316 | 7.6 | mmol⁴ | 230° C. | 50% | 98% |
| ELQ-400 | 7.8 | mmol⁴ | 250° C. | 32% | >99% |
| ELQ-400 | 7.3 | mmol⁴ | 230° C. | 33% | >99% |
| ELQ-271 | 8.1 | mmol⁴ | 250° C. | 63% | >99% |

¹Yields are calculated from the originally used 15, as the intermediate Schiff bases 20a-d were used without purification or characterization in the subsequent cyclization step.
²Purity determined by HPLC.
³Yield and purity of recrystallized ELQ-300.
⁴Back-calculated from the weight of Schiff bases 20a-d used.

Next, the general utility of using the β-keto ester 15 as an intermediate for the synthesis of other ELQ compounds, including ELQ-271, ELQ-316, and ELQ-400 (Nilsen, et al., Sci Transl Med 2013, 5 (177), 177ra37; Nilsen et al., J Med Chem 2014, 57 (9), 3818-34; Stickles et al., Am J Trop Med Hyg 2015, 92 (6), 1195-201) was investigated. In all cases, the condensation of β-keto ester 15 with the corresponding aniline 19a, 19c and 19d, respectively, followed by thermal cyclization, afforded the desired quinolones in relatively good yield. In the case of ELQ-316, the reaction proceeded well using 115.2 g of β-keto ester 15 to form the corresponding Schiff base 20c followed by cyclization at 250° C. Similar to the case of ELQ-300, in the cyclization of 20c to form ELQ-316 only trace formation of the undesired regioisomer 21b was detected by GC-MS of the corresponding 4-chloro derivative. The copper content of this sample was below the limit of detection (<17 ppm), as with 12 obtained in Scheme 3 (<21 ppm), suggesting that removal of copper is not a concern in this reaction sequence.

At this early stage in development, it is difficult to obtain an accurate estimate of the cost of goods for the industrial production of ELQ-300 and ELQ-316. However, using ELQ-300 as an example, it is possible to compare the relative cost of the original synthesis (Scheme 2) to the new efficient synthesis (Scheme 7). On one hand, the original synthesis comprises 7 reaction steps and has a 36% yield over its longest linear sequence (5 reaction steps), which does not take into account the yield of the 2 non-linear steps. Additionally, it requires the use of a relatively expensive palladium catalyst and at least two chromatographic separations and a high vacuum distillation. On the other hand, the new efficient synthesis comprises 5 reaction steps and has a 41% overall yield. It requires no expensive reagents, such as palladium, and no chromatographic separations. Based on length, relative cost of reagents, and simplicity of purification, we estimate the cost of the new efficient synthesis of ELQ compounds to be 10-20% of the cost of the original synthesis.

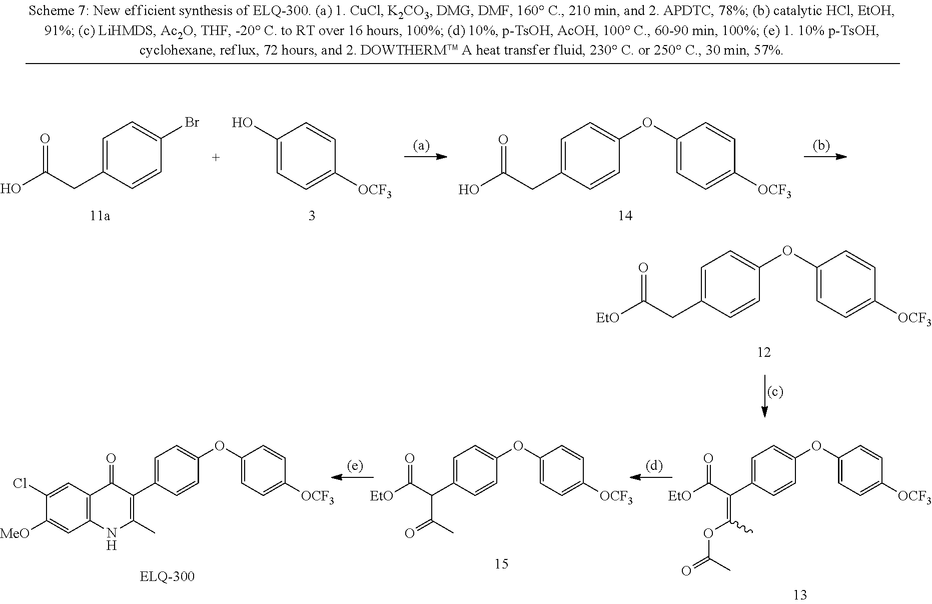

### Conclusions

The successful development of another efficient late-stage cyclization synthetic route to ELQ-300 and other structurally related ELQ derivatives (Scheme 7) is described in this Example; this synthetic route bypasses the more expensive palladium catalyzed Suzuki-Miyaura reaction originally developed by our lab to explore the initial structure-activity relationship of quinolone and side chain modifications to the core scaffold (Scheme 2) (Nilsen, et al., Sci Transl Med 2013, 5 (177), 177ra37; Nilsen et al., J Med Chem 2014, 57 (9), 3818-34). Additionally, we have found an efficient synthesis of a key intermediate diaryl ether 12 that does not require high vacuum distillation. The overall yield in this two-step synthesis (71%, Scheme 4) is better than the yield of the single step synthesis of 12 involving high vacuum distillation (48-60%) (Scheme 3). The key to the success of this efficient late-stage cyclization scheme is our ability to synthesize the bis-acylated product 13 and then to selectively convert it to the desired intermediate β-keto ester 15 using p-TsOH as a catalyst. This synthetic route is inexpensive and contains five scalable steps, with purification via a single recrystallization (as needed) and no chromatography or distillation. ELQ-300 and other ELQ compounds are obtained in pure form with an overall yield of ca. 30-40%. Further, the steps have not been fully optimized at industrial scale, and it is believed that improvements may be achieved by adjustment of reaction conditions (e.g., time and temperature), solvents, and equipment. Compared to the synthetic route for ELQ-300 and other ELQ compounds originally described by us (Nilsen, et al., Sci Transl Med 2013, 5 (177), 177ra37; Nilsen et al., J Med Chem 2014, 57 (9), 3818-34), this late-stage cyclization synthesis appears to be readily scalable and promises to considerably reduce the cost of goods. This is especially important in the case of ELQ-300 production given that it is the active component of the preclinical candidate antimalarial prodrug, ELQ-331, and the fact that malaria-endemic countries are among the world's most impoverished.

In addition to the advantages described above, this efficient synthetic route avoids the relatively harsh conditions (HBr in AcOH) required to deprotect the 4-O-ethyl ether quinoline intermediate in the previously described synthesis (Scheme 2) (Nilsen, et al., Sci Transl Med 2013, 5 (177), 177ra37; Nilsen et al., J Med Chem 2014, 57 (9), 3818-34). These conditions regularly resulted in the partial demethylation of the 7-OMe ether of both ELQ-300 and ELQ-316. Because the milder conditions of this new route do not result in demethylation of the 7-OMe ether, we are currently exploring using the new route to synthesize quinolone analogs with sensitive functional groups that were not accessible by the original route.

It is also noteworthy that ELQ-316 can be made efficiently and with high purity and yield by this new late-stage cyclization synthetic pathway. While ELQ-316 exhibits slightly less antimalarial activity than ELQ-300 in vivo, it has superior antiparasitic activity against a broader range of Apicomplexan protozoan species including Toxoplasma gondii and Babesia microti, which also cause severe and potentially fatal disease in humans. Recent studies show that both ELQ-300 and ELQ-316 are also active against a range of Apicomplexan parasites of importance to veterinary medicine (Silva et al., Parasit Vectors 2020, 13 (1), 606; Eberhard et al., Front Vet Sci 2020, 7, 96; Anghel et al., Front Vet Sci 2018, 5, 285).

### Methods

### Ethyl 2-(4-(4-(trifluoromethoxy)phenoxy)phenyl)acetate (12) from 11b and 3 (Scheme 3)

A round bottom flask, stir bar, and potassium carbonate were oven dried at 150° C. for at least 24 hours prior to use. Copper chloride (CuCl) (12.2 g, 123 mmol, 0.15 eq), DMG (8.5 g, 82.3 mmol, 0.1 eq), and dimethylformamide (DMF, 200 ml) were placed into the hot round bottom flask and degassed for 20 min at 50° C. under house vacuum, while stirring. To the intensely blue-colored catalyst mixture were added K₂CO₃ (227 g, 1.65 mol, 2.0 eq), ethyl-2-(4-bromophenyl)acetate (200 g, 0.823 mol, 1.0 eq) in 200 ml of degassed DMF, and 4-(trifluoromethoxy)phenol 3 (161.7 g, 0.908 mol, 1.1 eq). The reaction mixture was heated to 50° C., degassed for 10 min under house vacuum, and purged with argon for 5 min. The temperature was then raised and maintained at 160° C. for 90 minutes under argon, whereupon GC-MS analysis showed that 11b was consumed. Upon cooling, the solid residue was filtered, boiled with 500 ml ethyl acetate to extract the products from the residue, cooled, and then filtered again. This process was repeated one more time. All of the filtrates were combined and concentrated to give crude 12 as a black, oily product (355 g) that was purified by distillation under high vacuum (0.5-0.6 millitorr) at 140° C. to give 12 (134 g, 48% yield) as a yellow oil. GC-MS shows one major peak with M⁺=340 (37%), 267 (100%). ¹H-NMR (CDCl₃): δ 7.32-7.28 (m, 2H), 7.22-7.18 (m, 2H), 7.04-6.98 (m, 4H), 4.20 (q, J=7.1 Hz, 2H), 3.63 (s, 2H), 1.30 (t, J=7.1 Hz, 3H). Copper <21 ppm. HRMS calculated for C₁₇H₁₆F₃O₄[M+H]⁺=341.0995 observed for [M+H]⁺=341.0993.

### 2-(4-(4-(trifluoromethoxy)phenoxy)phenyl)acetic acid (14) from 11a and 3 (Scheme 4)

A round bottom flask, stir bar, and K₂CO₃ were oven dried at 150° C. for at least 24 hours, and the DMF was degassed under house vacuum for 1 hour prior to use. Copper (I) chloride (CuCl) (1.38 g, 13.9 mmol, 0.15 eq), DMG (0.96 g, 9.30 mmol, 0.10 eq), and DMF (30 ml) were placed into the hot round bottom flask and degassed for 20 min at 50° C., while stirring. To the blue-colored catalyst mixture DMF (100 ml) and K₂CO₃ (38.5 g, 279 mmol, 3.0 eq) were added. Next, 2-(4-bromophenyl)acetic acid (11a, 20 g, 93.0 mmol, 1.0 eq) was slowly added to avoid excessive foaming, followed by 4-(trifluoromethoxy)phenol (3, 19.9 g, 111.6 mmol, 1.2 eq). The reaction mixture was heated to 50° C., degassed for 10 min, and purged with argon for 5 min. The temperature was then raised and maintained at 160° C. for 3.5 hours under argon, when 2-(4-bromophenyl)acetic acid was consumed as shown by GC-MS analysis. The crude mixture was cooled to room temperature, and 200 ml water and ammonium pyrrolidinedithiocarbamate (APDTC) (5.0 g, 30.6 mmol, 2.2 eq wrt CuCl used) were added followed by stirring at 50° C. for 1 hour. The resulting slurry was passed through a Celite pad (50 g) and washed thoroughly with water (250 ml). To the filtrate was added ice (150 g) and concentrated HCl (12.1N, 55 ml) until the pH was around 2. The light yellow solid that precipitated out of solution was filtered, washed with hot water (100 ml), boiled in 300 ml water, cooled with 200 g ice, filtered again, and air dried. The above treatment with water was used to remove water soluble residues and some of the remaining phenol 3. The resulting 2-(4-(4-(trifluoromethoxy)phenoxy)phenyl)acetic acid 14 (22.7 g, 78% yield) was used in the subsequent step without further purification. GC-MS showed one major peak with M⁺=312 (53%), 267 (100%). ¹H-NMR (400 MHz; CDCl₃): δ 7.29 (d, J=8.4 Hz, 2H), 7.20 (dd, J=9.1, 0.8 Hz, 2H), 7.04-6.99 (m, 4H), 3.67 (s, 2H). Copper <73 ppm. HRMS calculated for C₁₅H₁₁F₃O₄ [M+H]⁺=313.0682, observed for [M+H]⁺=313.0678, and calculated for C₁₅H₁₁F₃O₄ [M+Na]⁺=335.0504, observed for [M+Na]⁺=335.0497. The product was at least 95% pure by GC-MS and ¹H-NMR. The only impurity was the phenol starting material 3.

### Ethyl 2-(4-(4-(trifluoromethoxy)phenoxy)phenyl)acetate (12) from 14 (Scheme 4)

Into a round bottom flask equipped with a stir bar were added 14 (22.6 g, 72.4 mmol, 1.0 eq), absolute ethanol (167 g, 3.6 mol, 50 eq) and 12.1 N HCl (0.75 mL, 9.1 mmol, 0.13 eq). The solution was heated to reflux for 18 hours, at the end of which time only ˜1% of the starting material 14 was present. The solvent was removed in vacuo, and the resulting thick slurry was passed through a silica gel plug (70 g), rinsing with a mixture of hexanes/ethyl acetate (3:1) until no more 12 came through as monitored by TLC. After solvent removal, ethyl 2-(4-(4-(trifluoromethoxy)phenoxy)phenyl)acetate 12 was recovered (22.5 g, 91% yield) as a light orange oil. GC-MS showed one major peak with M⁺=340 (37%), 267 (100%). ¹H-NMR (CDCl₃): δ 7.32-7.28 (m, 2H), 7.22-7.18 (m, 2H), 7.04-6.98 (m, 4H), 4.20 (q, J=7.1 Hz, 2H), 3.63 (s, 2H), 1.30 (t, J=7.1 Hz, 3H). Copper <12 ppm. HRMS calculated for C₁₇H₁₆F₃O₄ [M+H]⁺=341.0995, observed for [M+H]⁺=341.0993. The product was at least 95% pure by GC-MS and ¹H-NMR, sufficient for use in the subsequent step without further purification.

### General Procedure for the Preparation of the Schiff Bases (20a-d, Scheme 6)

A stirred mixture of substituted anilines 19a-d and β-ketoester 15 containing 10% of p-TsOH in cyclohexane was refluxed for 24-72 hours using a Dean-Stark trap to continuously remove the water formed during the condensation. After cooling to room temperature, the mixture was filtered and solid thus removed (containing unreacted anilines 19a-d and some unidentified materials) was washed with cyclohexane. The filtrate combined with the cyclohexane washes was concentrated in vacuo to give the products 20a-d as yellow-brown, highly viscous oils, which were used without purification in the next phase of the reaction.

### General Procedure for the Conrad-Limpach Reaction (ELQ, Scheme 6)

(Conrad & Limpach, Berichte der deutschen chemischen Gesellschaft 1887, 20 (1), 944-948; Conrad & Limpach, Berichte der deutschen chemischen Gesellschaft 1891, 24 (2), 2990-2992): The Conrad-Limpach reactions were performed at different temperatures. The 250° C. cyclization was conducted at the boiling point of DOWTHERM™ A heat transfer fluid. For other temperatures, the cyclization was conducted in DOWTHERM™ A heat transfer fluid maintained at the desired temperature with an internal thermometer. To facilitate addition of the highly viscous Schiff base obtained above, it was diluted with DOWTHERM™ A heat transfer fluid with slight warming. This mixture was added to the heated DOWTHERM™ A heat transfer fluid over 10 min with vigorous stirring, so that the desired temperature was always maintained. After stirring for a further period of time (specified for individual reactions below) the reaction mixture was cooled to room temperature and diluted with hexanes, resulting in the formation of a white precipitate. The precipitate was recovered by filtration, washed with hexanes, and air dried to give the crude ELQ compound. Yields were determined over two steps based on the β-ketoester 15 used to form the initial Schiff base.

### 2-Methyl-3-(4-(4-(trifluoromethoxy)phenoxy)phenyl)quinolin-4(1H)-one (ELQ-271)

### 1-Ethoxy-3-(phenylimino)-2-(4-(4-(trifluoromethoxy)phenoxy)phenyl)but-1-en-1-ol (20a, Scheme 6)

Following the general procedure for the preparation of the Schiff base a mixture of aniline 19a (1.85 g, 19.9 mmol, 1.0 eq), 8.07 g β-ketoester 15 containing 10% of p-TsOH (7.69 g, 20.1 mmol, 1.0 eq of 15 and 0.38 g, 2.6 mmol, 0.1 eq of p-TsOH) in cyclohexane (200 ml) was refluxed for 24 hours. Cyclohexane (30 ml) was used to wash the precipitate. The filtrate was concentrated in vacuo to give the crude Schiff base 20a (9.60 g) as a yellow, highly viscous oil.

Conrad-Limpach cyclization of 20a: A portion of the Schiff base 20a (3.90 g) was diluted with DOWTHERM™ A heat transfer fluid (5 ml) with slight warming. This Schiff base solution was added over 5 min to boiling DOWTHERM™ A heat transfer fluid (100 ml, 250° C.) with vigorous stirring, so that boiling was always maintained. After stirring for another 20 min at 250° C., the stirred reaction mixture was cooled to room temperature resulting in the formation of a white precipitate. When cold, hexanes (150 ml) was added to the mixture. It was then filtered, washed with hexanes (30 ml), acetone (100 ml) and air-dried to give crude ELQ-271 (2.11 g, 63%) as a white solid. ¹H-NMR (400 MHz; DMSO-d₆): δ 11.66 (s, 1H), 8.10-8.08 (m, 1H), 7.64 (ddd, J=8.4, 6.9, 1.5 Hz, 1H), 7.54 (ddd, J=8.3, 1.1, 0.6 Hz, 1H), 7.44-7.41 (m, 2H), 7.32-7.28 (m, 3H), 7.19-7.15 (m, 2H), 7.10-7.07 (m, 2H), 2.27 (s, 3H). HPLC analysis indicated that the obtained ELQ-271 was >99% pure. GC-MS analysis of the 4-chloro derivative of ELQ-271 obtained by chlorination by POCl₃ showed only a few very small uncharacterized impurities besides the major component with M⁺=429.5.

### 6-Chloro-7-methoxy-2-methyl-3-(4-(4-(trifluoromethoxy)phenoxy)phenyl)quinolin-4(1H)-one (ELQ-300)

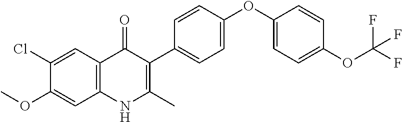 [SMILES: COc1cc2[nH]c(C)c(-c3ccc(Oc4ccc(OC(F)(F)F)cc4)cc3)c(=O)c2cc1Cl]

### 3-(4-chloro-3-methoxyphenyl)imino)-1-ethoxy-2-(4-(4-(trifluoromethoxy)phenoxy)-phenyl)but-1-en-1-ol (20b)

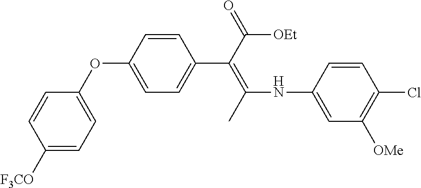 [SMILES: CCOC(=O)/C(=C(/C)Nc1ccc(Cl)c(OC)c1)c1ccc(Oc2ccc(OC(F)(F)F)cc2)cc1]

Following the general procedure for the preparation of the Schiff base a mixture of 4-chloro-3-methoxyaniline 19b (20.63 g, 131 mmol, 1.0 eq.), 52.25 g of ketoester 15 containing 10 mol-% of p-TsOH (50.0 g, 131.0 mmol, 1.0 eq of 15 and 2.25 g, 1.31 mmol, 0.1 eq of p-TsOH) in cyclohexane (300 ml) was refluxed for 44 hours. Cyclohexane (100 ml) was used to wash the precipitate. The condensation was practically complete after 20 hours, as no substantial increase in the volume of water of condensation occurred after this time. The combined filtrates were concentrated in vacuo to give the crude Schiff base 20b (67.5 g) as a yellow, highly viscous oil.

Conrad-Limpach cyclization of 20b: The Schiff base 20b (67.5 g) was diluted with DOWTHERM™ A heat transfer fluid (30 ml) with slight warming. This Schiff base 20b solution was added in small portion over 25 min to boiling DOWTHERM™ A heat transfer fluid (550 ml, 250° C.) with vigorous stirring, so that boiling was always maintained. After stirring for another 10 min at 250° C. the stirred reaction mixture was cooled to room temperature resulting in the formation of a thick white precipitate. The mixture was then allowed to stand without stirring at room temperature overnight. Hexanes (1000 ml) was added and the mixture was stirred for 15 min and filtered. The resulting white precipitate was washed with ethyl acetate (500 ml) and acetone (250 ml) and air dried to give ELQ-300 (35.5 g, 57% yield). ¹H-NMR (400 MHz; DMSO-d₆): δ 11.66 (s, 1H), 8.00 (s, 1H), 7.43-7.41 (m, 2H), 7.30-7.26 (m, 2H), 7.18-7.14 (m, 2H), 7.09-7.05 (m, 3H), 3.97 (s, 3H), 2.24 (s, 3H). HPLC analysis indicated that ELQ-300 was >99% pure. No regioisomer 21a was observed by ¹H-NMR, and GC-MS analysis of the 4-chloro derivative of ELQ-300 obtained by chlorination with POCl₃ showed only a single compound to be present, M⁺=493.1.

To assess the performance of the Conrad-Limpach reaction at temperatures below 250° C. using the Schiff base 20b, 10.0 g of β-ketoester 15 containing 10 mol-% of p-TsOH (9.5 g, 25 mmol, 1.0 eq of 14 and 0.5 g, 2.9 mmol, 0.1 eq of p-TsOH) and 1.0 equivalents of aniline 19b (4.0 g, 25 mmol) were condensed as described above, and the cold cyclohexane solution was filtered and diluted to a total volume of 300 ml. From this stock solution (Solution A), aliquots of specific volume were removed, stripped of solvent, diluted with DOWTHERM™ A heat transfer fluid and cyclised at the specified temperatures.

The cyclization at 230° C. was performed similarly to that at 250° C., above. To 100 ml of rapidly stirred DOWTHERM™ A heat transfer fluid held at 230° C., the residue of a 100 mL aliquot of Solution A (corresponding to 8.3 mmols of 15 condensed with 19b) was dissolved in DOWTHERM™ A heat transfer fluid (10 ml) and added dropwise over the course of 10 min. The temperature was carefully monitored, and the heat reservoir was sufficiently large to keep temperature changes within 1° C. After stirring for another 20 min at 230° C., the stirred reaction mixture was cooled to room temperature resulting in the formation of a white precipitate. The mixture was filtered, washed with hexanes and acetone and air dried to give crude ELQ-300 (1.88 g, 48% yield) as a white solid. As in the case of the cyclization at 250° C., no regioisomer 21a was observed by ¹H-NMR and GC-MS analysis of the 4-chloro derivative of ELQ-300 obtained by chlorination with POCl₃. The ¹H-NMR spectrum of ELQ-300 obtained from reaction at 230° C. was identical to that of ELQ-300 obtained at 250° C. HPLC analysis indicated that the obtained ELQ-300 was >99% pure.

The cyclization at 200° C. was performed similarly to the above. To 100 ml of rapidly stirred DOWTHERM™ A heat transfer fluid held at 200° C., the residue of a 22 ml aliquot of Solution A (corresponding to 1.8 mmol condensed 15) in DOWTHERM™ A heat transfer fluid (5 ml) was added dropwise over the course of 10 min. The temperature was carefully monitored and the heat reservoir was sufficiently large to keep temperature changes within 1° C. After stirring for another 60 min at 200° C., the stirred reaction mixture was allowed to cool to room temperature and let stand overnight, resulting in the formation of a white precipitate. Hexanes (100 ml) was added and the mixture was filtered, washed with hexanes, and air-dried to give ELQ-300 (0.26 g, 30% yield) as a white solid. As in the cases of the cyclizations at 250° C. and at 230° C. no regioisomer 21a was observed by ¹H-NMR and GC-MS analysis of the 4-chloro derivative of ELQ-300 obtained by chlorination with POCl₃. The ¹H-NMR spectrum of ELQ-300 obtained from reaction at 200° C. was identical to that of ELQ-300 obtained at 250° C. HPLC analysis indicated that the obtained ELQ-300 was >99% pure.

### 6-Fluoro-7-methoxy-2-methyl-3-(4-(4-(trifluoromethoxy)phenoxy)phenyl)quinolin-4(1H)-one (ELQ-316)

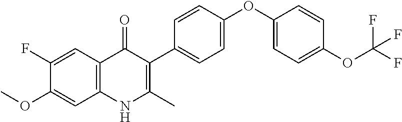 [SMILES: COc1cc2[nH]c(C)c(-c3ccc(Oc4ccc(OC(F)(F)F)cc4)cc3)c(=O)c2cc1F]

### 1-Ethoxy-3-((4-fluoro-3-methoxyphenyl)imino)-2-(4-(4-(trifluoromethoxy)-phenoxy)phenyl)but-1-en-1-ol (20c)

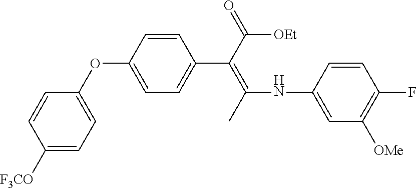 [SMILES: CCOC(=O)/C(=C(/C)Nc1ccc(F)c(OC)c1)c1ccc(Oc2ccc(OC(F)(F)F)cc2)cc1]

Following the general procedure for the preparation of the Schiff base a mixture of 4-fluoro-3-methoxyaniline 19c (42.5 g, 0.301 mol, 1 eq), 120.7 g β-ketoester 15 containing 10% of p-TsOH (115.2 g, 0.302 mol, 1.0 eq of 15 and 5.5 g, 0.1 eq of p-TsOH) in cyclohexane (600 ml) was refluxed for 72 hours. Cyclohexane (50 ml) was used to wash the precipitate. The combined filtrates were concentrated in vacuo to give the crude Schiff base 20c as a yellow, highly viscous oil.

Conrad-Limpach cyclization of 20c: The Schiff base 20c was diluted with DOWTHERM™ A heat transfer fluid (50 ml) with slight warming. This Schiff base solution was added over 30 min to boiling DOWTHERM™ A heat transfer fluid (700 ml, 250° C.) with vigorous stirring, so that boiling was always maintained. After stirring for another 15 min at 250° C., the reaction mixture was cooled to room temperature, resulting in the formation of a firm, white cake. Ethyl acetate (2 I) was added and the cake broken up with a glass rod and stirred until no large pieces remained. The mixture was filtered, washed with methanol (500 ml), then with acetone (250 ml) and air-dried to give crude ELQ-316 (73 g, 54%) as a white solid. ¹H-NMR (400 MHz; DMSO-d₆): β 11.63 (s, 1H), 7.70 (d, J=11.8 Hz, 1H), 7.43-7.40 (m, 2H), 7.30-7.26 (m, 2H), 7.18-7.14 (m, 2H), 7.11-7.05 (m, 3H), 3.96 (s, 3H), 2.24 (s, 3H). HPLC analysis indicated that the obtained ELQ-316 was >99% pure. Copper <17 ppm. No regioisomer 21b was observed by ¹H-NMR, and GC-MS analysis of the 4-chloro derivative of ELQ-316 obtained by chlorination with POCl₃ showed only a single compound to be present, M⁺=477.5.

The cyclization at 230° C. was performed similarly to the above. To 100 ml of rapidly stirred DOWTHERM™ A heat transfer fluid held at 230° C., 3.84 g of the condensation product 20c in DOWTHERM™ A heat transfer fluid (2 ml) was added dropwise over the course of 10 minutes. The temperature was carefully monitored, and the heat reservoir was sufficiently large to keep temperature changes within 1° C. After stirring for another 30 minutes at 230° C., the stirred reaction mixture was cooled to room temperature resulting in the formation of a white precipitate. Hexanes (100 ml) was then added to the mixture. It was then filtered, washed with hexanes (30 ml) and ethyl acetate (2×10 ml) and air-dried to give ELQ-316 (1.75 g, 50%) as a white solid. No regioisomer 21b was observed by ¹H-NMR and GC-MS analysis of the 4-chloro derivative of ELQ-316 obtained by chlorination with POCl₃. The ¹H-NMR spectrum of ELQ-316 obtained from reaction at 230° C. was identical to that of ELQ-316 obtained at 250° C. HPLC analysis indicated that the obtained ELQ-316 was 98% pure.

### 5.7-Difluoro-7-methoxy-2-methyl-3-(4-(4-(trifluoromethoxy)phenoxy)phenyl)quinolin-1H)-one (ELQ-400)

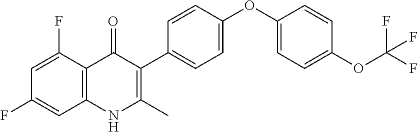 [SMILES: Cc1[nH]c2cc(F)cc(F)c2c(=O)c1-c1ccc(Oc2ccc(OC(F)(F)F)cc2)cc1]

### 1-ethoxy-3-((3,5-difluorophenyl)imino)-2-(4-(4(trifluoromethoxy)phenoxy)phenyl) but-1-en-1-ol (20d)

Following the general procedure for the preparation of the Schiff base, a mixture of 3,5-difluoro aniline 19d (3.26 g, 25.3 mmol, 1.0 eq), 10.14 g β-ketoester 15 containing 10% of p-TsOH (9.66 g, 25.3 mmol, 1.0 eq of 15 and 0.48 g, 2.8 mmol, 0.1 eq of p-TsOH) in cyclohexane (200 ml) was refluxed for 24 hours. After cooling to room temperature, the clear solution was separated from the residue stuck to the walls of the reaction flask and was concentrated in vacuo to give the crude Schiff base 20d (13.0 g) as a yellow, highly viscous oil.

Conrad-Limpach cyclization of 20d: A portion of the Schiff base 20d (4.00 g) was diluted with DOWTHERM™ A heat transfer fluid (2 ml) with slight warming. The Schiff base solution was added over 5 min to boiling DOWTHERM™ A heat transfer fluid (90 ml, 250° C.) with vigorous stirring, so that boiling was always maintained. After stirring for another 15 min at 250° C. the stirred reaction mixture was cooled to room temperature, resulting in the formation of a white precipitate. When cold, hexanes (100 ml) was added to the mixture. After brief stirring it was filtered, washed with hexanes (30 ml) and air-dried to give crude ELQ-400 (1.90 g, 32%) as a white solid. ¹H-NMR (400 MHz; DMSO-d6): β 11.74 (s, 1H), 7.43-7.40 (m, 2H), 7.28-7.25 (m, 2H), 7.18-7.14 (m, 2H), 7.10-7.00 (m, 4H), 2.21 (s, 3H). HPLC analysis indicated that the obtained ELQ-400 was >99% pure.

The cyclization at 230° C. was performed similarly to the above. To 100 ml of rapidly stirred DOWTHERM™ A heat transfer fluid held at 230° C., 3.80 g of the condensation product 20d in DOWTHERM™ A heat transfer fluid (5 ml) was added dropwise over the course of 10 min. The temperature was carefully monitored, and the heat reservoir was sufficiently large to keep temperature changes within 1° C. After stirring for another 30 min at 230° C., the stirred reaction mixture was cooled to room temperature resulting in the formation of a white precipitate. Hexanes (150 ml) was then added, and the precipitate was collected by filtration, washed with hexanes (50 ml) and acetone (10 ml) and air-dried to give crude ELQ-400 (1.14 g, 33%) as a slightly yellow solid. The ¹H-NMR spectrum of ELQ-400 obtained from reaction at 230° C. was identical to that of ELQ-400 obtained at 250° C. HPLC analysis indicated that the obtained ELQ-400 was >99% pure.

### ADDITIONAL LITERATURE CITATIONS

Cross et al., J Med Chem. 53(1):7076-94, 2010
Gallagher et al., Org. Proc. Res. Devel., 23(6): 1269-1274, 2019

### Closing Paragraphs

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in the production of a target ELQ compound or intermediate herein, or in the efficacy of that compound or intermediate.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles, other written text, and web site content throughout this specification (referenced materials herein). Each of the referenced materials are individually incorporated herein by reference in their entirety for their referenced teaching as of the filing date of the first application in the priority chain in which the specific reference was included.

It will be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the example(s) or when application of the meaning renders any construction meaningless or essentially meaningless. In cases where the construction of the term would render it meaningless or essentially meaningless, the definition should be taken from Webster's Dictionary, 11th Edition or a relevant dictionary known to those of ordinary skill in the art, such as the Oxford Dictionary of Biochemistry and Molecular Biology, 2^{nd} Edition (Ed. Anthony Smith, Oxford University Press, Oxford, 2006), and/or A Dictionary of Chemistry, 8^{th} Edition (Ed. J. Law & R. Rennie, Oxford University Press, 2020).

What is claimed:

1. A method for the preparation of a compound of Formula (I): 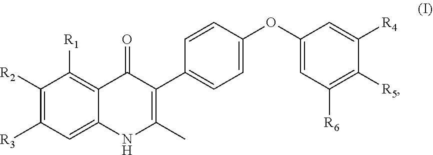
wherein:
R₁ is selected from the group of H, F, Cl, Br, CN, —CH₂F, —CHF₂, —CF₃, —OCH₃, —OCH₂F, —OCHF₂, —OCF₃, —SF₅, and —CH₃;
R₂ is selected from the group of H, F, Cl, Br, CN, —CH₂F, —CHF₂, —CF₃, —OCH₃, —OCH₂F, —OCHF₂, —OCF₃, —SF₅, and —CH₃;
R₃ is selected from the group of H, F, Cl, Br, CN, —CH₂F, —CHF₂, —CF₃, —OCH₃, —OCH₂F, —OCHF₂, —OCF₃, —SF₅, and —CH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃;
the method comprising the steps of:
a) reacting an optionally substituted phenol compound of Formula (A) 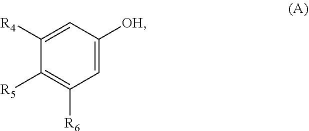
wherein R₄, R₅, and R₆ are as defined above, with an alkyl 2-(4-bromophenyl) acetate ester compound of the Formula (B) 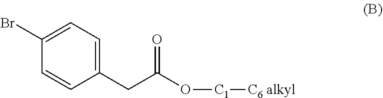
to produce an optionally substituted alkyl 2-(4-phenoxyphenyl)acetate ester compound of Formula (C) 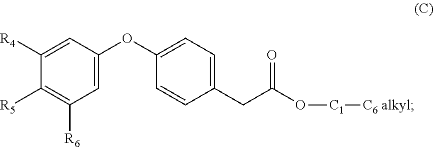
b) treating the compound of Formula (C) with a non-nucleophilic base and acetic anhydride to prepare a compound of Formula (D) 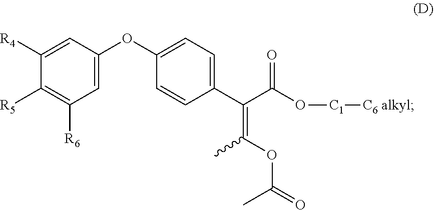
c) treating the compound of Formula (D) with an acid to prepare a compound of Formula (E) 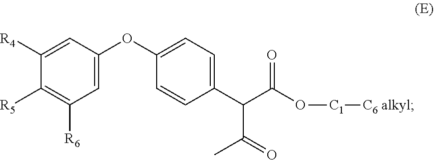
d) reacting the compound of Formula (E) with an optionally substituted aniline compound of Formula (F) 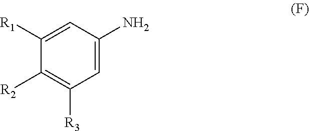
to prepare a compound of Formula (G) 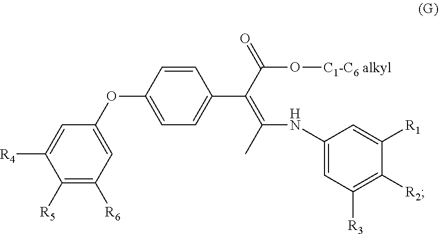
and
e) heating the compound of Formula (G) at a temperature of from about 150° C. to about 300° C. to prepare the compound of Formula (I).

2. The method of claim 1, wherein:
R₁ is selected from the group of H and F;
R₂ is selected from the group of H, F, and Cl;
R₃ is selected from the group of H F, and —OCH₃; and
R₄, R₅, and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃.

3. A method of preparing a compound of Formula (C): 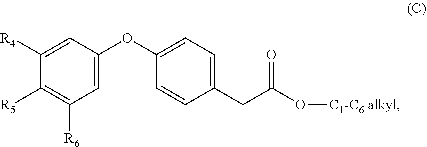
the method comprising reacting an optionally substituted phenol compound of Formula (A) 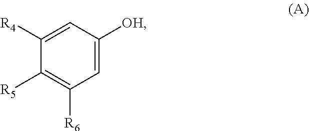
wherein R₄, R₅, and R₆ are as defined above, with an alkyl 2-(4-bromophenyl) acetate compound of the Formula (B)

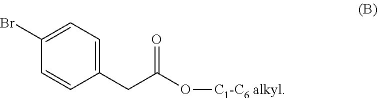

4. A method for the preparation of compound of Formula (D): 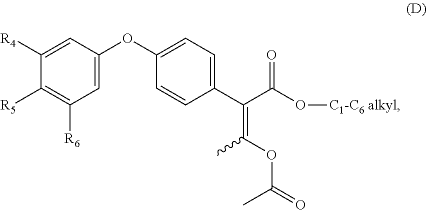
the method comprising treating the compound of Formula (C), above, with a non-nucleophilic base and acetic anhydride.

5. A method of preparing a compound of Formula (E): 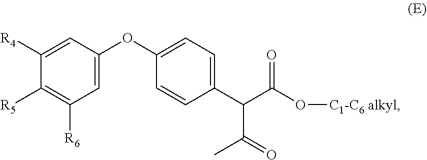
the method comprising reacting a compound of Formula (D), 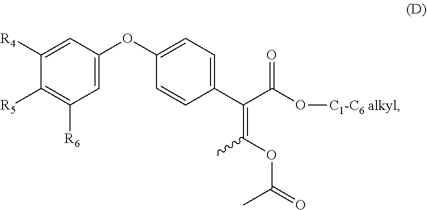
with an acid.

6. A method to prepare a compound of Formula (G): 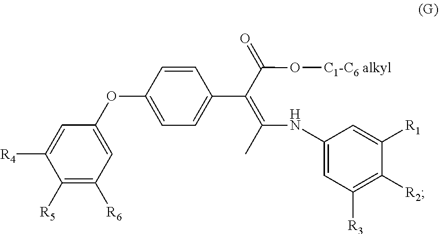
wherein:
R₁ is selected from the group of H and F;
R₂ is selected from the group of H, F, and Cl; and
R₃ is selected from the group of H, F, and —OCH₃;
the method comprising reacting the compound of Formula (E), 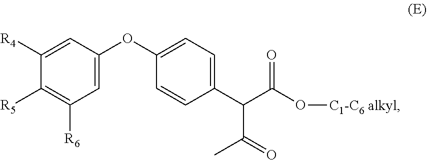
with an optionally substituted aniline compound of Formula (F)

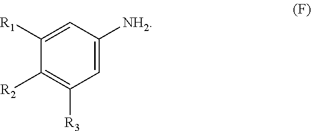

7. A method of preparing a compound of Formula (I); 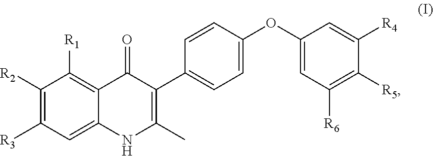
wherein: R₁ is selected from the group of H, F, Cl, Br, CN, —CH₂F, —CHF₂, —CF₃, —OCH₃, —OCH₂F, —OCHF₂, —OCF₃, —SF₅, and —CH₃; R₂ is selected from the group of H, F, Cl, Br, CN, —CH₂F, —CHF₂, —CF₃, —OCH₃, —OCH₂F, —OCHF₂, —OCF₃, —SF₅, and —CH₃; R₃ is selected from the group of H, F, Cl, Br, CN, —CH₂F, —CHF₂, —CF₃, —OCH₃, —OCH₂F, —OCHF₂, —OCF₃, —SF₅, and —CH₃; R₄ is selected from the group of H, F, Cl, Br, CN, —CH₂F, —CHF₂, —CF₃, —OCH₃, —OCH₂F, —OCHF₂, —OCF₃, —SF₅, and —CH₃; R₅ is selected from the group of H, F, Cl, Br, CN, —CH₂F, —CHF₂, —CF₃, —OCH₃, —OCH₂F, —OCHF₂, —OCF₃, —SF₅, and —CH₃; and R₆ is selected from the group of H, F, Cl, Br, CN, —CH₂F, —CHF₂, —CF₃, —OCH₃, —OCH₂F, —OCHF₂, —OCF₃, —SF₅, and —CH₃;
the method comprising treating a compound of Formula (G) 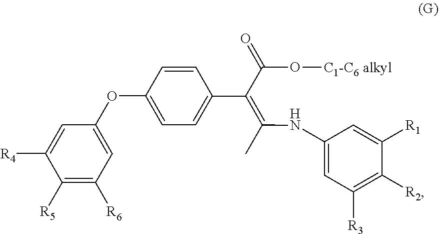
to a temperature of from about 150° C. to about 300° C. for a period of time effective to prepare the compound of Formula (I).

8. The method of claim 7, wherein:
R₁ is selected from the group of H and F;
R₂ is selected from the group of H, F, and Cl;
and each of R₃, R₄, R₅, and R₆ is independently selected from the group of H, F, Cl, Br, CN, —CH₂F, —CHF₂, —CF₃, —OCH₃, —OCH₂F, —OCHF₂, —OCF₃, —SF₅, and —CH₃.

9. The method of claim 7, wherein:
R₁ is selected from the group of H and F;
R₂ is selected from the group of H, F, and Cl;
R₃ is selected from the group of H, F, and —OCH₃;
and each of R₄, R₅, and R₆ is independently selected from the group of H, F, Cl, Br, CN, —CH₂F, —CHF₂, —CF₃, —OCH₃, —OCH₂F, —OCHF₂, —OCF₃, —SF₅, and —CH₃.

10. A compound of Formula (C-1): 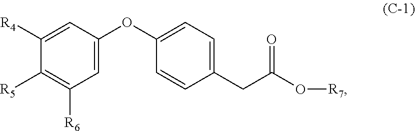
wherein R₄, R₅, and R₆ are each independently selected from the group of H, F, Cl, Br, CN, —CH₂F, —CHF₂, —CF₃, —OCH₂F, —OCHF₂, —OCF₃, —SF₅, and —CH₃; and R₇ is C₁-C₆ alkyl; with the proviso that, when R₇ is methyl, at least one of R₄, R₅, and R₆ is not H.

11. The compound of claim 7, wherein R₅ is —OCF₃, R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

12. The compound of claim 10 or claim 11, wherein R₅ is —OCF₃, R₄ and R₆ are each H; and R₇ is C₁-C₆ alkyl.

13. The compound of any of claims 10-12, which has the structure:

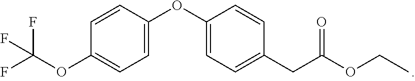 [SMILES: CCOC(=O)Cc1ccc(Oc2ccc(OC(F)(F)F)cc2)cc1]

14. A compound of Formula (D-1): 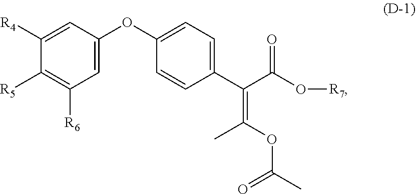
wherein: R₄, R₅, and R₆ are each independently selected from the group of H, F, Cl, Br, CN, —OCH₃, —CH₂F, —CHF₂, —CF₃, —SF₅, and —CH₃; and R₇ is C₁-C₆ alkyl.

15. The compound of claim 14, wherein R₅ is —OCF₃, R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

16. The compound of claim 14 or claim 15, wherein R₅ is —OCF₃, R₄ and R₆ are each H; and R₇ is C₁-C₆ alkyl.

17. The compound of any of claims 14-16, wherein R₅ is —OCF₃, R₄ and R₆ are each H; and R₇ is C₁-C₃ alkyl.

18. The compound of any of claims 14-17 having the structure:

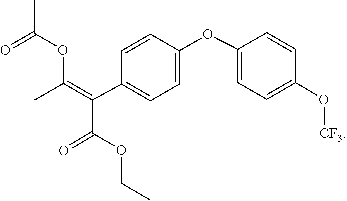 [SMILES: CCOC(=O)/C(=C(\C)OC(C)=O)c1ccc(Oc2ccc(OC(F)(F)F)cc2)cc1]

19. A compound of the Formula (E-1): 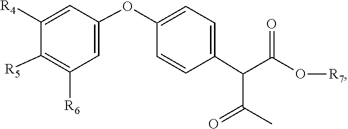
wherein: R₄, R₅, and R₆ are each independently selected from the group of H, F, Cl, Br, CN, —OCH₃, —CH₂F, —CHF₂, —CF₃, —SF₅, and —CH₃; and R₇ is C₁-C₆ alkyl; with the proviso that, when R₇ is methyl, at least one of R₄, R₅, and R₆ is not H.

20. The compound of claim 19, wherein R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₆ alkyl.

21. The compound of claim 19 or claim 2-, wherein R₅ is —OCF₃; R₄ and R₆ are each H; and R₇ is C₁-C₆ alkyl.

22. The compound of any of claims 19-21, wherein R₅ is —OCF₃; R₄ and R₆ are each H; and R₇ is C₁-C₃ alkyl.

23. The compound of any of claims 19-21, wherein R₅ is —OCF₃; R₄ and R₆ are each H; and R₇ is C₁-C₂ alkyl.

24. A compound of Formula (G-1): 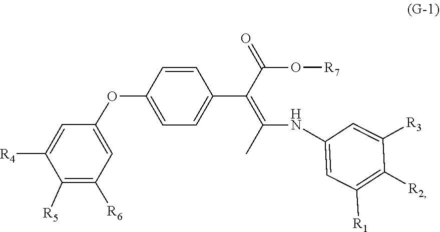
wherein:
R₁ is selected from the group of H and F;
R₂ is selected from the group of H, F, and Cl;
R₃ is selected from the group of H, F, and —OCH₃;
R₄, R₅, and R₆ are each independently selected from the group of H, F, Cl, Br, CN, —OCH₃, —CH₂F, —CHF₂, —CF₃, —SF₅, and —CH₃; and
R₇ is C₁-C₆ alkyl.

25. The compound of claim 23, wherein R₁ is selected from the group of H and F; R₂ is selected from the group of H, F, and Cl; R₃ is selected from the group of H, F, and —OCH₃; R₅ is —OCF₃; R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₃ alkyl.

26. The compound of claim 23, wherein R₁ is selected from the group of H and F; R₂ is selected from the group of H, F, and Cl; R₃ is selected from the group of H, F, and —OCH₃; R₅ is —OCF₃, R₄ and R₆ are each independently selected from the group of H, F, Cl, Br, CN, —CH₂F, —CHF₂, —CF₃, —OCH₃, —OCH₂F, —OCHF₂, —OCF₃, —SF₅, and —CH₃; and R₇ is C₁-C₂ alkyl.

27. The compound of claim any one of claims 23-26 wherein R₁ is selected from the group of H and F; R₂ is selected from the group of H, F, and Cl; R₃ is selected from the group of H, F, and —OCH₃; R₅ is —OCF₃, R₄ and R₆ are each H; and R₇ is C₁-C₃ alkyl.

28. The compound of any one of claims 25-27, wherein R₁ is selected from the group of H and F; R₂ is selected from the group of H, F, and Cl; R₃ is selected from the group of H, F, and —OCH₃; R₅ is —OCF₃, R₄ and R₆ are each H; and R₇ is C₁-C₂ alkyl.

29. The compound of any of claims 24-28, selected from the group of:

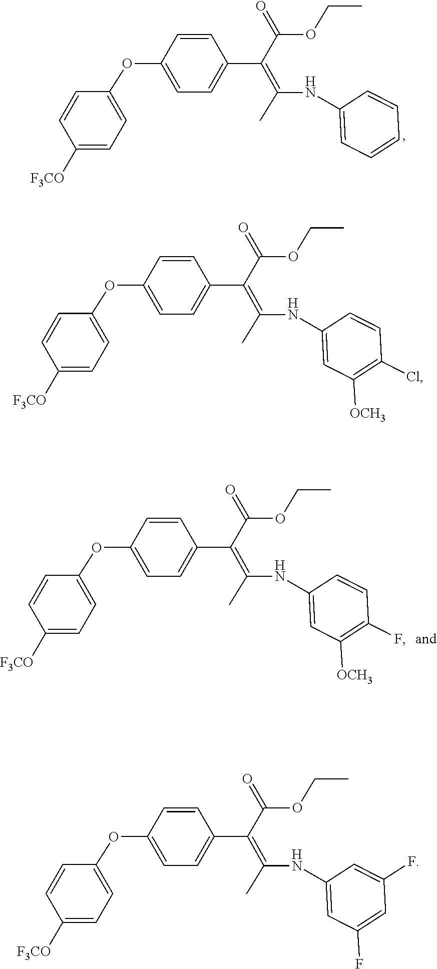 [SMILES: CCOC(=O)/C(=C(/C)Nc1ccccc1)c1ccc(Oc2ccc(OC(F)(F)F)cc2)cc1 ; CCOC(=O)/C(=C(/C)Nc1ccc(Cl)c(OC)c1)c1ccc(Oc2ccc(OC(F)(F)F)cc2)cc1 ; CCOC(=O)/C(=C(/C)Nc1ccc(F)c(OC)c1)c1ccc(Oc2ccc(OC(F)(F)F)cc2)cc1 ; CCOC(=O)/C(=C(/C)Nc1cc(F)cc(F)c1)c1ccc(Oc2ccc(OC(F)(F)F)cc2)cc1]

30. A compound of Formula (C-1a): 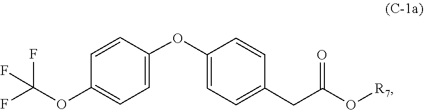
wherein R₇ is C₁-C₆ alkyl.

31. The compound of claim 30, wherein R₇ is C₁-C₄ alkyl.

32. The compound of compound of claim 30 or claim 31, wherein R₇ is C₁-C₃ alkyl.

33. The compound of compound of claim 30 or claim 31, wherein R₇ is C₁-C₂ alkyl.

34. The compound of any of claims 30-33, which is ethyl 2-(4-(4-(trifluoromethoxy)phenoxy)phenyl)acetate.

35. A method for the preparation of a compound of Formula (I): 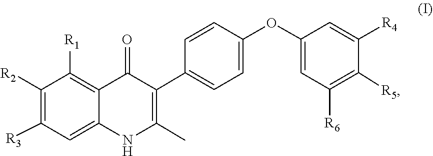
wherein:
R₁ is selected from the group of H, F, Cl, Br, CN, —CH₂F, —CHF₂, —CF₃, —OCH₃, —OCH₂F, —OCHF₂, —OCF₃, —SF₅, and —CH₃;
R₂ is selected from the group of H, F, Cl, Br, CN, —CH₂F, —CHF₂, —CF₃, —OCH₃, —OCH₂F, —OCHF₂, —OCF₃, —SF₅, and —CH₃;
R₃ is selected from the group of H, F, Cl, Br, CN, —CH₂F, —CHF₂, —CF₃, —OCH₃, —OCH₂F, —OCHF₂, —OCF₃, —SF₅, and —CH₃;
R₄ is selected from the group of H, F, Cl, Br, CN, —CH₂F, —CHF₂, —CF₃, —OCH₃, —OCH₂F, —OCHF₂, —OCF₃, —SF₅, and —CH₃;
R₅ is selected from the group of H, F, Cl, Br, CN, —CH₂F, —CHF₂, —CF₃, —OCH₃, —OCH₂F, —OCHF₂, —OCF₃, —SF₅, and —CH₃; and
R₆ is selected from the group of H, F, Cl, Br, CN, —CH₂F, —CHF₂, —CF₃, —OCH₃, —OCH₂F, —OCHF₂, —OCF₃, —SF₅, and —CH₃;
the method comprising the steps of:
a) reacting an optionally substituted phenol compound of Formula (A) 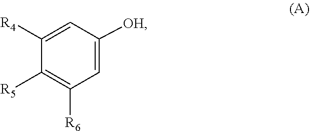
wherein R₄, R₅, and R₆ are as defined above, with 4-bromophenylacetic acid, in the presence of a copper catalyst, to form intermediate diaryl ether carboxylic acid (H) 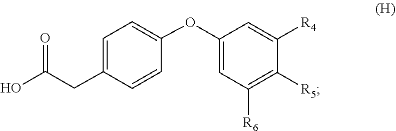
b) removing the copper catalyst using APDTC;
c) esterifying the diaryl ether carboxylic acid (16) to form an optionally substituted alkyl 2-(4-phenoxyphenyl)acetate compound of Formula (C) 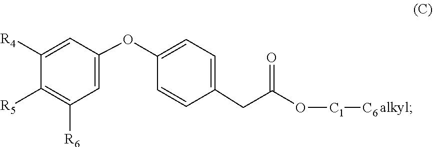
d) treating the compound of Formula (C) with a non-nucleophilic base and acetic anhydride to prepare a compound of Formula (D) 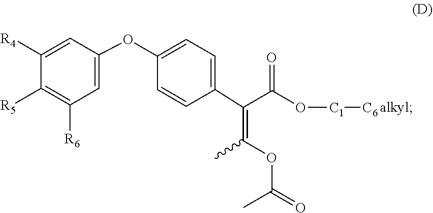
e) treating the compound of Formula (D) with an acid to prepare a compound of Formula (E) 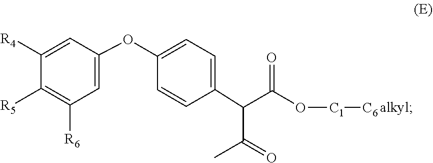
f) reacting the compound of Formula (E) with an optionally substituted aniline compound of Formula (F) 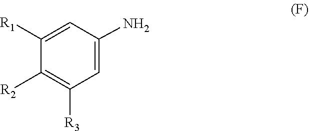
to prepare a compound of Formula (G) 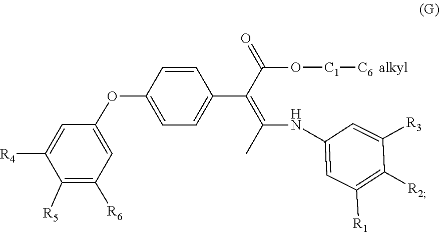
and
g) heating the compound of Formula (G) at a temperature of from about 150° C. to about 300° C. to prepare the compound of Formula (I).

36. A method of preparing a compound of Formula (C): 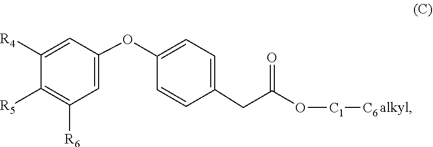
the method comprising:
reacting, in the presence of a copper catalyst, 4-bromophenylacetic acid (11a) with a phenol moiety substituted by R₄, R₅, and R₆, as defined herein, to form the intermediate diaryl ether carboxylic acid (H);
removing the copper catalyst; and
esterifying the carboxylic acid (H) to Compound (C).

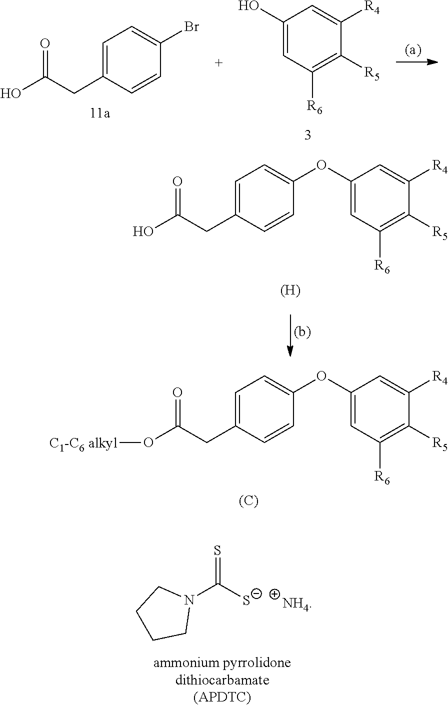

37. A method of preparing Compound (C), comprising:
e) reacting 4-bromophenylacetic acid (11a) with a phenol moiety substituted by R₄, R₅, and R₆ (3), as defined herein, in a first medium comprising a copper catalyst to form a second medium comprising the intermediate diaryl ether carboxylic acid (H) and the copper catalyst;
f) removing the copper catalyst from the second medium with a copper chelating agent to create a third medium comprising the intermediate diaryl ether carboxylic acid (16); and
g) reacting the intermediate diaryl ether carboxylic acid (H) in the third medium with a C₁-C₆ alkanol to form esterified Compound (C).

38. The method of claim 37, wherein the copper chelating agent a dithiocarbamate chelating agent, such as ammonium pyrrolidine dithiocarbamate (APDTC), sodium dimethyldithiocarbamate (NaDMDTC), or sodium diethyldithiocarbamate (NaDEDTC).

39. The method of claim 37, wherein the copper chelating agent is ammonium pyrrolidine dithiocarbamate (APDTC).

40. The method of any one of claims 35-39, wherein the C₁-C₆ alkanol is ethanol.
